(19) United States
(12) Patent Application Publication
(10) Pub. No.: US 2012/0135989 A1
(43) Pub. Date: May 31, 2012

(54) AZAINDOLE DERIVATIVE

(75) Inventors: Yoko Funakoshi, Tokyo (JP); Chika Tanaka, Tokyo (JP); Choul Hong Park, Seoul (KR); Seong Gu Ro, Seoul (KR)

(73) Assignees: CRYSTALGENOMICS, INC., Seoul (KR); SBI BIOTECH CO., LTD., Tokyo (JP)

(21) Appl. No.: 13/172,007

(22) Filed: Jun. 29, 2011

Related U.S. Application Data
(60) Provisional application No. 61/359,613, filed on Jun. 29, 2010.

(51) Int. Cl. A61K 31/5377; C07D 487/04; A61P 35/00; A61K 31/496; A61K 31/519; C07D 471/04; A61K 31/437

(52) U.S. Cl. 514/234.5; 546/113; 544/362; 544/127; 544/280; 514/300; 514/253.04; 514/265.1

(57) ABSTRACT

The present invention provides a (di)azaindole derivative represented by the formula (I). A compound of the present invention inhibits a Cdc7 protein kinase activity and suppresses cell proliferation.

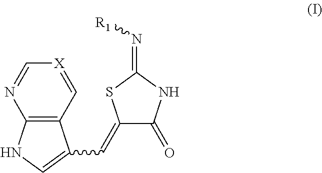

### TECHNICAL FIELD

The present invention relates to azaindole derivatives, diazaindole derivatives, geometric isomers and tautomers thereof, as well as salts, hydrates, or solvates thereof.

### BACKGROUND ART

In general, the biological function of proteins is modulated by various mechanisms of post-translational modifications. Specifically, it has been shown that methylation, acetylation, glycosylation, phosphorylation, or the like, is involved in modifications of functions or structures of proteins. Among these post-translational modifications, phosphorylation is an important mechanism related to modulation of many functions such as intracellular signal transduction, cell cycle, cell death, or the like. For example, it is thought that one-third or more of the intracellular proteins of mammalian cells are phosphorylated.

Proteins are phosphorylated by the action of protein kinases. In general, protein kinases catalyze a reaction of bonding a phosphate group to a specific site of a specific substrate protein. That is to say, proteins are phosphorylated on specific amino acid residues. Thus, protein kinases can be classified as follows based on amino acids at a site to be phosphorylated.

Serine—threonine kinase (Ser/S or Thr/T residue is phosphorylated)

Tyrosine kinase (Tyr/Y is phosphorylated)

Human Cdc7 that is one of the serine-threonine kinases is a protein kinase involved in the start of DNA replication in a cell cycle. Specifically, it is thought that with phosphorylation of MCM (Minichromosome maintenance) protein by Cdc7, Cdc45 and DNA polymerase are recruited by DNA and the DNA replication starts. The phosphorylation action of Cdc7 needs a cofactor. For example, ASK is identified as a cofactor that activates the phosphorylation action of Cdc7.

It is thought that Cdc7 involved in DNA replication can be an important target for cell proliferation diseases such as cancers. In other words, when the DNA replication necessary for the cell proliferation can be controlled by inhibiting Cdc7, the cell proliferation may be suppressed. Various compounds, which have an inhibitory action of protein kinase such as Cdc7, have been reported to date (PATENT DOCUMENTS 1 to 5).

### CITATION LIST

### Patent Documents

PATENT DOCUMENT 1: WO2007/071621
PATENT DOCUMENT 2: WO2007/096334
PATENT DOCUMENT 3: WO2007/110344
PATENT DOCUMENT 4: WO2007/124288
PATENT DOCUMENT 5: WO2008/046982

### SUMMARY OF INVENTION

### Problems to be Solved by the Invention

An object of the present invention is to provide novel azaindole derivatives and diazaindole derivatives (hereinafter, which together are also referred to as “(di)azaindole derivatives”), geometric isomers and tautomers thereof, as well as salts, hydrates, or solvates thereof. Alternatively, the present invention provides (di)azaindole derivatives having a Cdc7 protein kinase inhibitory action. Alternatively, the present invention provides (di)azaindole derivatives having a suppressing action on cell proliferation.

### Means for Solving the Problems

The present invention provides (di)azaindole derivatives represented by the following formula (I), geometric isomers and tautomers thereof as well as salts, hydrates, or solvates thereof. Furthermore, the present invention provides a production process of (di)azaindole derivatives represented by the following formula (I).

[1] A compound represented by the following formula (I), a geometric isomer or a tautomer thereof, or a salt, a hydrate, or a solvate thereof:

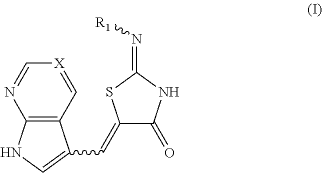

wherein

### X is CH or N;

R₁ is selected from the group consisting of a straight or branched chain lower alkyl group, a cycloalkyl group that may have a substituent, an aryl group that may have a substituent, an arylalkyl group that may have a substituent, a non-aromatic heterocyclic group that may have a substituent, and a heteroaryl group that may have a substituent, or is a condensed ring group that may have a substituent; and a wavy line, independently for each occurrence, denotes trans (E-form), cis (Z-form) or a mixture (mixed product) thereof.

[2] The compound, a geometric isomer and a tautomer thereof, as well as a salt, a hydrate, or a solvate thereof described in [1], wherein R₁ is an aryl group or a heteroaryl group which may be substituted with one to three groups independently selected from the following Group B;

Group B: a straight or branched chain lower alkyl group which may be substituted with a group selected from the group consisting of one to three halogen atoms, a hydroxyl group, an amino group substituted with one or two lower alkyl groups and a non-aromatic heterocyclic group;

a lower alkoxy group;

a hydroxyl group;

a halogen group;

a nitro group;

an amino group that may be substituted with one or two lower alkyl groups;

a lower alkylcarbonylamino group;

a group represented by a formula: —(CH₂)ₖCOOH, wherein k is 0 to 2;

a group represented by a formula: —O—R₂—R₃, wherein R₂ is a single bond, a lower alkylene group or a cycloalkylene group, or a non-aromatic heterocyclic group that may be substituted with a lower alkyl group; and R₃ is a group selected from a hydroxyl group; a carboxyl group; a lower alkoxy group; a lower alkoxycarbonyl group; an amino group substituted with two lower alkyl groups or with one lower alkyl group and one lower alkoxy carbonyl group; and a non-aromatic heterocyclic group that may be substituted with a lower alkyl group; and

a group represented by a formula: —CON(R₄)[(CH₂)ₘ—R₅], wherein m is 0 to 2, R₄ is a hydrogen atom or a lower alkyl group, and R₅ is an amino group that is substituted with one or two lower alkyl groups.

[3] The compound, a geometric isomer and a tautomer thereof, as well as a salt, a hydrate, or a solvate thereof described in [2], wherein the aryl group or the heteroaryl group is a phenyl group, a naphthyl group, an indolyl group, an indazolyl group, a quinolyl group, a benzimidazolyl group or a benzotriazolyl group.

[4] The compound, a geometric isomer and a tautomer thereof, as well as a salt, a hydrate, or a solvate thereof described in [1], wherein the R₁ is a benzyl group having a substituent, and a benzene ring of the benzyl group is substituted with halogen, a lower alkyl group that may be substituted with one to three halogen atoms or a lower alkoxy group, or methylene of the benzyl group is substituted with one or two lower alkyl groups.

[5] The compound, a geometric isomer and a tautomer thereof, as well as a salt, a hydrate, or a solvate thereof described in [1], wherein the R₁ is an indanyl group or a 1,3-benzodioxolyl group.

[6] The compound, a geometric isomer and a tautomer thereof, as well as a salt, a hydrate, or a solvate thereof described in [1], wherein the R₁ is a phenyl group having a substituent.

[7] The compound, a geometric isomer and a tautomer thereof, as well as a salt, a hydrate, or a solvate thereof described in [1], wherein the R₁ is a phenyl group having a substituent, and the substituent is a group represented by a formula: —O—R₂—R₃, wherein R₂ is a single bond, a lower alkylene group or a cycloalkylene group, or a non-aromatic heterocyclic group that may be substituted with a lower alkyl group; and R₃ is a group selected from a hydroxyl group; a carboxyl group; a lower alkoxy group; a lower alkoxycarbonyl group; an amino group substituted with two lower alkyl groups, or with one lower alkyl group and one lower alkoxy carbonyl group; and a non-aromatic heterocyclic group that may be substituted with a lower alkyl group.

[8] A pharmaceutical composition including a compound, a geometric isomer and a tautomer thereof, or a salt, a hydrate, or a solvate thereof described in [1] to [7], and a pharmaceutically acceptable carrier.

[9] A production process of a compound represented by the following formula (I), which includes reacting a compound represented by the following formula (IIA) or (IIB) with a compound represented by the following formula (III):

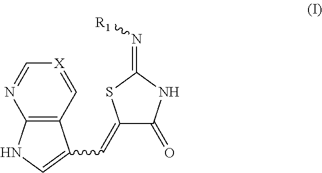

wherein R₁ is selected from the group consisting of a straight or branched chain lower alkyl group, a cycloalkyl group that may have a substituent, an aryl group that may have a substituent, an arylalkyl group that may have a substituent, a non-aromatic heterocyclic group that may have a substituent, and a heteroaryl group that may have a substituent, or is a condensed ring group that may have a substituent; and a wavy line, independently for each occurrence, denotes trans (E-form), cis (Z-form) or a mixture (mixed product) thereof.

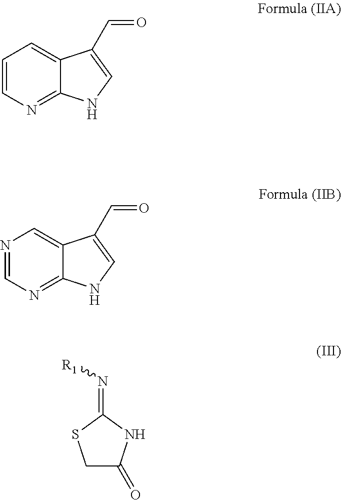

wherein R₁ is the same as the R₁ in the above-mentioned formula (I).

[10] The compound, a geometric isomer and a tautomer thereof, or a salt, a hydrate, or a solvate thereof described in [1], selected from the group consisting of (2Z,5Z)-2-(4-methoxy-2-methylphenyl)azamethylene-5-(1H-pyrrolo[2,3-b]pyridin-3-yl)methylene-1,3-thiazolidin-4-one, (2Z,5Z)-2-(2,4-dimethylphenyl)azamethylene-5-(1H-pyrrolo[2,3-b]pyridin-3-yl)methylene-1,3-thiazolidin-4-one, (2Z,5Z)-2-(4-hydroxy-2-methylphenyl)azamethylene-5-(1H-pyrrolo[2,3-b]pyridin-3-yl)methylene-1,3-thiazolidin-4-one, (2Z,5Z)-2-(4-carboxyphenyl)azamethylene-5-(1H-pyrrolo[2,3-b]pyridin-3-yl)methylene-1,3-thiazolidin-4-one, (2Z,5Z)-2-{4-(2-dimethylamino)ethoxy-2-methylphenyl}azamethylene-5-(1H-pyrrolo[2,3-b]pyridin-3-yl)methylene-1,3-thiazolidin-4-one, (2Z,5Z)-2-{4-(1-methylpyrrolidin-3-yl)oxy-2-methylphenyl}azamethylene-5-(1H-pyrrolo[2,3-b]pyridin-3-yl)methylene-1,3-thiazolidin-4-one, (2Z,5Z)-2-[4-{2-(4-methylpiperazin-1-yl)}ethoxy-2-methylphenyl]azamethylene-5-(1H-pyrrolo[2,3-b]pyridin-3-yl)methylene-1,3-thiazolidin-4-one, (2Z,5Z)-2-{4-(1-methyl-piperidin-4-yl)oxy-2-methylphenyl}azamethylene-5-(1H-pyrrolo[2,3-b]pyridin-3-yl)methylene-1,3-thiazolidin-4-one, (2Z,5Z)-2-{4-(2-morpholino)ethoxy-2-methylphenyl}azamethylene-5-(1H-pyrrolo[2,3-b]pyridin-3-yl)methylene-1,3-thiazolidin-4-one, (2Z,5Z)-2-{2-chloro-4-(2-dimethylamino)ethoxyphenyl}azamethylene-5-(1H-pyrrolo[2,3-b]pyridin-3-yl)methylene-1,3-thiazolidin-4-one, (2Z,5Z)-2-{4-(2-dimethylamino)ethoxy-2-methylphenyl}azamethylene-5-(7H-pyrrolo[2,3-d]pyrimidin-5-yl)methylene-1,3-thiazolidin-4-one, (2Z,5Z)-2-{2-chloro-4-(1-methylpyrrolidin-3-yl)oxyphenyl}azamethylene-5-(1H-pyrrolo[2,3-b]pyridin-3-yl)methylene-1,3-thiazolidin-4-one, (2Z,5Z)-2-{4-(2-dimethylamino)ethoxy-2-trifluoromethylphenyl}azamethylene-5-(1H-pyrrolo[2,3-b]pyridin-3-yl)methylene-1,3-thiazolidin-4-one, (2Z,5Z)-2-{4-(1-methylpyrrolidin-3-yl)oxy-2-trifluoromethylphenyl}azamethylene-5-(1H-pyrrolo[2,3-b]pyridin-3-yl)methylene-1,3-thiazolidin-4-one, (2Z,5Z)-2-{4-(1-methylpyrrolidin-3-yl)oxy-2-methylphenyl}azamethylene-5-(7H-pyrrolo[2,3-d]pyrimidin-5-yl)methylene-1,3-thiazolidin-4-one, (2Z,5Z)-2-{4-(1-methyl-piperidin-4-yl)oxy-2-methylphenyl}azamethylene-5-(7H-pyrrolo[2,3-d]pyrimidin-5-yl)methylene-1,3-thiazolidin-4-one, (2Z,5Z)-2-{2-chloro-4-(2-dimethylamino)ethoxyphenyl}azamethylene-5-(7H-pyrrolo[2,3-d]pyrimidin-5-yl)methylene-1,3-thiazolidin-4-one, (2Z,5Z)-2-{2-chloro-4-(1-methylpyrrolidin-3-yl)oxyphenyl}azamethylene-5-(7H-pyrrolo[2,3-d]pyrimidin-5-yl)methylene-1,3-thiazolidin-4-one, (2Z,5Z)-2-{4-(2-dimethylamino)ethoxy-2-trifluoromethylphenyl}azamethylene-5-(7H-pyrrolo[2,3-d]pyrimidin-5-yl)methylene-1,3-thiazolidin-4-one, (2Z,5Z)-2-{4-(1-methylpyrrolidin-3-yl)oxy-2-trifluoromethylphenyl}azamethylene-5-(7H-pyrrolo[2,3-d]pyrimidin-5-yl)methylene-1,3-thiazolidin-4-one, (2Z,5Z)-2-{2-chloro-4-(1-methyl-piperidin-4-yl)oxyphenyl}azamethylene-5-(7H-pyrrolo[2,3-d]pyrimidin-5-yl)methylene-1,3-thiazolidin-4-one, (2Z,5Z)-2-{2-chloro-4-(1-methyl-piperidin-4-yl)oxyphenyl}azamethylene-5-(1H-pyrrolo[2,3-b]pyridin-3-yl)methylene-1,3-thiazolidin-4-one and (2Z,5Z)-2-{4-(1-methyl-piperidin-4-yl)oxy-2-trifluoromethylphenyl}azamethylene-5-(1H-pyrrolo[2,3-b]pyridin-3-yl)methylene-1,3-thiazolidin-4-one.

### Advantages of the Invention

The present invention provides novel (di)azaindole derivatives. Compounds provided by the present invention are useful as a Cdc7 protein kinase inhibitor. A Cdc7 protein kinase is an enzyme that is closely involved in a cell cycle, particularly in the start of DNA replication.

Therefore, the compound having a Cdc7 protein kinase inhibitory action, which is provided by the present invention, can suppress cell proliferation. It has been confirmed that the (di)azaindole derivatives of the present invention exhibit a strong cell proliferation suppression action.

### DESCRIPTION OF EMBODIMENTS

Hereinafter, the meaning of terms, marks, and the like, used in the present specification are described, and the present invention is described in detail.

The term “lower” used herein means a group having one to eight carbon atoms, preferably one to seven carbon atoms, more preferably one to six carbon atoms, further preferably one to five carbon atoms and still further preferably one to four carbon atoms, unless otherwise specified.

The term “may have a substituent” used herein means that one or two or more of any types of substituents may be included in any chemically possible positions, unless otherwise specified. When two or more types of substituents are present, they may be the same as each other or may be different from each other.

The term “alkyl group” used herein denotes a monovalent group derived by removing any one hydrogen atom from aliphatic hydrocarbon that contains no heteroatom or no unsaturated carbon-carbon bond in the skeleton. Specific examples of the “lower alkyl group” include an alkyl group having one to six carbon atoms (C₁₋₆ alkyl group). More specific examples include a methyl group, an ethyl group, a 1-propyl group, an isopropyl group, an n-butyl group, an s-butyl group, a t-butyl group, an isobutyl group, a 1-pentyl group, a 2-pentyl group, a 3-pentyl group, a 2-methyl-1-butyl group, a 3-methyl-1-butyl group, a 2-methyl-2-butyl group, a 3-methyl-2-butyl group, a 2,2-dimethyl-1-propyl group, a 1-hexyl group, a 2-hexyl group, a 3-hexyl group, a 2-methyl-1-pentyl group, a 3-methyl-1-pentyl group, a 4-methyl-1-pentyl group, a 2-methyl-2-pentyl group, a 3-methyl-2-pentyl group, a 4-methyl-2-pentyl group, a 2-methyl-3-pentyl group, a 3-methyl-3-pentyl group, a 2,3-dimethyl-1-butyl group, a 3,3-dimethyl-1-butyl group, a 2,2-dimethyl-1-butyl group, a 2-ethyl-1-butyl group, a 3,3-dimethyl-2-butyl group, and a 2,3-dimethyl-2-butyl group.

The term “lower alkylene group” used herein denotes a divalent group derived by further removing any one hydrogen atom from the above-defined “lower alkyl group.”

Specific examples of the lower alkylene group include an alkylene group having one to six carbon atoms (C₁₋₆ alkylene group). More specific examples include a methylene group, an ethylene group, a trimethylene group, a propylene group, a tetramethylene group, a pentamethylene group, and a hexamethylene group.

The term “lower alkoxy group” used herein denotes an oxy group to which the above-defined “lower alkyl group”, is bonded.

Specific examples of the “lower alkoxy group” include an alkoxy group having one to six carbon atoms (C₁₋₆ alkoxy group), and more specific examples include a methoxy group, an ethoxy group, a 1-propyloxy group, a 2-propyloxy group, 2-methyl-1-propyloxy group, a 2-methyl-2-propyloxy group, a 1-butyloxy group, a 2-butyloxy group, 1-pentyloxy group, a 2-pentyloxy group, a 3-pentyloxy group, a 2-methyl-1-butyloxy group, a 3-methyl-1-butyloxy group, a 2-methyl-2-butyloxy group, 3-methyl-2-butyloxy group, a 2,2-dimethyl-1-propyloxy group, a 1-hexyloxy group, a 2-hexyloxy group, a 3-hexyloxy group, a 2-methyl-1-pentyloxy group, a 3-methyl-1-pentyloxy group, a 4-methyl-1-pentyloxy group, a 2-methyl-2-pentyloxy group, a 3-methyl-2-pentyloxy group, a 4-methyl-2-pentyloxy group, a 2-methyl-3-pentyloxy group, a 3-methyl-3-pentyloxy group, a 2,3-dimethyl-1-butyloxy group, a 3,3-dimethyl-1-butyloxy group, a 2,2-dimethyl-1-butyloxy group, a 2-ethyl-1-butyloxy group, a 3,3-dimethyl-2-butyloxy group, and a 2,3-dimethyl-2-butyloxy group.

The term “lower alkoxycarbonyl group” used herein denotes a carbonyl group to which the above-defined “lower alkoxy group” is bonded. Specific examples of the “lower alkoxycarbonyl group” include a carbonyl group to which a C₁₋₆ alkoxy group is bonded (C₁₋₆ alkoxycarbonyl group). More specific examples include a methoxycarbonyl group, an ethoxycarbonyl group, a 1-propyloxycarbonyl group, and a 2-propyloxycarbonyl group.

The term “lower alkylcarbonylamino group” used herein denotes an amino group to which the carbonyl group to which the above-defined “lower alkyl group” is bonded is bonded.

Specific examples of the lower alkylcarbonylamino group include an alkylcarbonylamino group having 1 to 6 carbon atoms (C₁₋₆ alkylcarbonylamino group), and more specific examples include an acetylamino group, a propionylamino group, and a butyrylamino group.

The term “halogen group” used herein denotes a fluorine atom, a chlorine atom, a bromine atom, or an iodine atom.

The term “lower alkyl group substituted with one to three halogen atoms” used herein denotes a lower alkyl group in which the same or different one to three “halogens” are bonded to the “lower alkyl group” (which is also referred to as a lower halogenated alkyl group).

Specific examples of the lower alkyl group substituted with one to three halogen atoms include a trifluoromethyl group, a trichloromethyl group, a difluoromethyl group, a dichloromethyl group, a dibromomethyl group, a fluoromethyl group, a 2,2,2-trifluoroethyl group, a 2,2,2-trichloroethyl group, 2-bromoethyl group, a 2-chloroethyl group, a 2-fluoroethyl group, a 2-iodoethyl group, a pentafluoroethyl group, a 3-chloropropyl group, a 4-fluorobutyl group, a 6-iodohexyl group, and a 2,2-dibromoethyl group.

The term “alkyl group substituted with a hydroxyl group” used herein denotes a group in which any hydrogen atom in the above-defined “alkyl group” is substituted with a hydroxyl group.

Specific examples of such a group include a hydroxymethyl group, a 2-hydroxyethyl group, a 1-hydroxyethyl group, a 3-hydroxy propyl group, a 2-hydroxy propyl group, a 1-hydroxy propyl group, and a 4-hydroxy butyl group.

The term “cycloalkyl group” used herein denotes a monovalent group derived by removing any one hydrogen atom from a cyclic saturated hydrocarbon ring.

Specific examples of the cycloalkyl group include a cycloalkyl group having three to eight carbon atoms (C₃₋₈ cycloalkyl group), and more specific examples include a cyclopropyl group, a cyclobutyl group, a cyclopentyl group, a cyclohexyl group, a cycloheptyl group, and a cyclooctyl group.

The term “cycloalkylene group” used herein denotes a divalent group derived by further removing any one hydrogen atom from the above-defined “cycloalkyl group.”

Specific examples of the cycloalkylene group include a cycloalkylene group having three to eight carbon atoms (C₃₋₈ cycloalkylene group), and more specific examples include a cyclopropylene group, a cyclobutylene group, a cyclopentylene group, and a cyclohexylene group.

The term “alkyl group substituted with a cycloalkyl group” used herein denotes a group in which any hydrogen atom in the above-identified “alkyl group” is replaced by the above-identified “cycloalkyl group”.

Specific examples of such a group include a cyclopropyl methyl group, a cyclobutyl methyl group, a cyclopentyl methyl group, a cyclohexyl methyl group, a cycloheptyl methyl group, a cyclooctyl methyl group, a 1-cyclopropyl ethyl group, a 2-cyclopropyl ethyl group, a 1-cyclobutyl ethyl group, a 2-cyclobutyl ethyl group, a 1-cyclopentyl ethyl group, a 2-cyclopentyl ethyl group, a 1-cyclohexyl ethyl group, a 2-cyclohexyl ethyl group, a 1-cycloheptyl ethyl group, a 2-cycloheptyl ethyl group, a 1-cyclooctyl ethyl group, a 2-cyclooctyl ethyl group, and a cyclopropylpropyl group.

The term “aryl group” used herein denotes an aromatic hydrocarbon cyclic group. Specific examples of the aryl group include an aryl group having six to ten carbon atoms (C₆₋₁₀ aryl group), and more specific examples include a phenyl group, a 1-naphthyl group, and a 2-naphthyl group.

The term “arylalkyl group” used herein denotes a group in which any hydrogen atom in the above-defined “alkyl group” is replaced by the above-defined “aryl group.”

Specific examples of the arylalkyl group include a C₆₋₁₀ aryl C₁₋₅ alkyl group, and more specific examples include a benzyl group, a phenethyl group, and a 3-phenyl-1-propyl group.

The term “non-aromatic heterocyclic group” used herein denotes a monocyclic or polycyclic non-aromatic monovalent group that includes one to three heteroatoms (sulfur, oxygen and nitrogen atoms) in the atoms constituting a ring, and may include a double bond in the ring.

Specific examples of the non-aromatic heterocyclic group include five- to seven-membered ring non-aromatic heterocyclic group (5- to 7-membered non-aromatic heterocyclic group). More specific examples include a pyrrolidinyl group, a dihydropyrrolyl group, an imidazolidinyl group, a pyrazolidinyl group, an oxazolidinyl group, a thiazolidinyl group, a piperidyl group, a dihydropyridinyl group, a tetrahydropyridinyl group, a dihydropyrimidinyl group, a tetrahydropyrimidinyl group, a hexahydropyrimidinyl group, a 1,3-oxadinyl group, a pyranyl group, a dihydropyranyl group, a tetrahydropyranyl group, a tetrahydrofuranyl group, a dihydrofuranyl group, a piperazinyl group, a morpholinyl group, a thiomorpholinyl group, and a 1,3-dioxolanyl group.

The term “non-aromatic heterocyclic group substituted with a lower alkyl group” used herein denotes a group in which any one of the hydrogen atoms of the above-defined “non-aromatic heterocyclic group” is replaced by the above-defined “lower alkyl group.”

Specific examples of such a “non-aromatic heterocyclic group substituted with a lower alkyl group” include a 1-methylpyrrolidin-2-yl group, a 1-methylpyrrolidin-3-yl group, a 1-methylimidazolidin-2-yl group, a 1-methylimidazolidin-3-yl group, a 1-methylpyrazolidin-3-yl group, a 1-methylpyrazolidin-4-yl group, a 1-methylpiperidin-2-yl group, a 1-methylpiperidin-3-yl group, a 1-methylpiperidin-4-yl group, a 4-methylpiperazin-1-yl group, a 1-methylpiperazin-4-yl group, a 1-ethylpyrrolidin-2-yl group, a 1-ethylpyrrolidin-3-yl group, a 1-ethylimidazolidin-2-yl group, a 1-ethylimidazolidin-3-yl group, a 1-ethylpyrazolidin-3-yl group, a 1-ethylpyrazolidin-4-yl group, a 1-ethylpiperidin-2-yl group, a 1-ethylpiperidin-3-yl group, a 1-ethylpiperidin-4-yl group, a 4-ethylpiperazin-1-yl group, and a 1-ethylpiperazin-4-yl group.

The term “alkyl group substituted with a non-aromatic heterocyclic group” used herein denotes a group in which any hydrogen atom in the above-defined “alkyl group” is replaced by the above-defined “non-aromatic heterocyclic group.”

Specific examples of such a group include a morpholin-4-yl-methyl group, a 1-(morpholin-4-yl)ethyl group, a 2-(morpholin-4-yl)ethyl group, a pyrrolidin-1-ylmethyl group, a 1-(pyrrolidin-1-yl)ethyl group, a 2-(pyrrolidin-1-yl)ethyl group, a piperidin-1-ylmethyl group, a 1-(piperidin-1-yl)ethyl group, a 2-(piperidin-1-yl)ethyl group, and a 3-(piperidin-1-yl)propyl group.

The term “heteroaryl group” used herein denotes a monovalent group derived from an aromatic ring including one or a plurality of heteroatoms (sulfur, oxygen and nitrogen atoms) in the atoms constituting a ring. The ring can be monocyclic or polycyclic.

Specific examples of the heteroaryl group include five- to ten-membered ring heteroaryl group (5- to 10-membered heteroaryl group). More specific examples include a pyridyl group, a thiophenyl group, a furanyl group, a pyrrolyl group, an oxazolyl group, an isoxazolyl group, a thiazolyl group, a thiadiazolyl group, an isothiazolyl group, an imidazolyl group, a triazolyl group, a pyrazolyl group, a furazanyl group, a thiadiazolyl group, an oxadiazolyl group, a pyridazinyl group, a pyrimidinyl group, a pyrazinyl group, a triazinyl group, an indolyl group, an isoindolyl group, an indazolyl group, a chromenyl group, a quinolyl group, an isoquinolyl group, a cinnolinyl group, a quinazolinyl group, a naphthyridinyl group, a phthalazinyl group, a purinyl group, a pteridinyl group, a thienofuranyl group, an imidazothiazolyl group, a benzofuranyl group, a benzothiophenyl group, a benzooxazolyl group, a benzothiazolyl group, a benzothiadiazolyl group, a benzimidazolyl group, a benzotriazolyl group, an imidazopyridinyl group, a pyrrolopyridinyl group, and a pyrrolopyrimidinyl group.

The term “condensed ring group” used herein denotes a monovalent group derived from a polycyclic compound in which “cycloalkane” and “arene” are condensed, or a polycyclic compound in which “non-aromatic heterocycle” and “arene” are condensed.

The “cycloalkane” denotes a cyclic saturated hydrocarbon ring, and specifically includes cycloalkane having three to eight carbon atoms. More specific examples include cyclopropane, cyclobutane, cyclopentane, cyclohexane, cycloheptane, and cyclooctane.

The “arene” denotes an aromatic hydrocarbon ring, and specifically includes arene having six to ten carbon atoms. More specific examples include benzene, and naphthalene.

The “non-aromatic heterocycle” denotes a monocyclic or polycyclic non-aromatic heterocycle that includes one to three heteroatoms (sulfur, oxygen and nitrogen atoms) in the atoms constituting the ring and that may have a double bond in the ring. Specific examples include five- to seven-membered ring non-aromatic heterocycle. More specific examples include pyrrolidine, dihydropyrrole, imidazolidine, pyrazolidine, oxazolidine, thiazolidine, piperidine, dihydropyridine, tetrahydropyridine, dihydropyrimidine, tetrahydropyrimidine, hexahydropyrimidine, 1,3-oxazine, pyrane, dihydropyrane, tetrahydropyrane, tetrahydrofuran, dihydrofuran, piperazine, morpholine, thiomorpholine, and 1,3-dioxolane.

Specific examples of such a condensed group includes an indanyl group, a 1,2,3,4-tetrahydronaphthyl group, a 3,4-dihydro-2H-1,4-benzooxadinyl group, a 3,4-dihydro-2H-1,4-benzothiazinyl group, a 1,3-benzodioxolyl group, a 2,3-dihydro-1.4-benzodioxinyl group, a chromanyl group, an isochromanyl group, a 3,4-dihydro-2H-1-benzothiopyranyl group, a 3,4-dihydro-1H-2-benzothiopyranyl group, an indolinyl group, an iosindolinyl group, a 1,2,3,4-tetrahydroquinolyl group, and a 1,2,3,4-tetrahydroisoquinolyl group.

The term “amino group substituted with one or two lower alkyl groups” used herein denotes an amino group in which a hydrogen atom(s) of the amino group is replaced by one or two of the “lower alkyl groups.”

Specific examples of a mono alkylamino group, in which a hydrogen atom of the amino group is substituted with one lower alkyl group, include a methylamino group, an ethylamino group, and a propylamino group.

Specific examples of a dialkylamino group, in which hydrogen atoms of the amino group are replaced by two lower alkyl groups, include a dimethyl amino group, a diethyl amino group, a methylethylamino group, and a methylpropylamino group.

The term “amino group substituted with one lower alkyl group and one lower alkoxycarbonyl group” used herein denotes an amino group in which hydrogen atoms of the amino group are replaced by the above-defined “lower alkyl group” and the above-defined “lower alkoxycarbonyl group.”

Specific examples of such a group include an N-methoxycarbonyl-N-methylamino group, an N-methoxycarbonyl-N-ethylamino group, an N-ethoxycarbonyl-N-methylamino group, and an N-methoxycarbonyl-N-ethylamino group.

The term “straight or branched chain lower alkyl group substituted with an amino group substituted with one or two lower alkyl groups” used herein denotes a group in which any hydrogen atom in the above-defined “lower alkyl group” is replaced by the above-defined “amino group substituted with one or two lower alkyl groups.”

Specific examples of such a group include a methylamino methyl group, a 1-(methylamino)ethyl group, a 2-(methylamino)ethyl group, a 1-(methylamino)propyl group, a 2-(methylamino)propyl group, a 3-(methylamino)propyl group, a dimethylaminomethyl group, a 1-(dimethylamino)ethyl group, a 2-(dimethylamino)ethyl group, a 1-(dimethylamino)propyl group, a 2-(dimethylamino)propyl group, a 3-(dimethylamino)propyl group, an ethylaminomethyl group, a 1-(ethylamino)ethyl group, a 2-(ethylamino)ethyl group, a 1-(ethylamino)propyl group, a 2-(ethylamino)propyl group, a 3-(ethylamino)propyl group, a diethylamino methyl group, a 1-(diethylamino)ethyl group, a 2-(diethylamino)ethyl group, a 1-(diethylamino)propyl group, a 2-(diethylamino)propyl group, and a 3-(diethylamino)propyl group.

The term “aminocarbonyl group substituted with one or two lower alkyl groups” used herein denotes a carbonyl group to which the above-defined “amino group substituted with one or two lower alkyl groups” is bonded.

Specific examples of such a group include a methylaminocarbonyl group, an ethylaminocarbonyl group, a propylaminocarbonyl group, a dimethylaminocarbonyl group, a diethylaminocarbonyl group, a methylethylaminocarbonyl group, and a methylpropylaminocarbonyl group.

The term “solvate” used herein denotes a group of molecules in which one or more types of solvent molecules and compounds are associated with each other in a stoichiometric manner or a non-stoichiometric manner.

Furthermore, the “hydrate” used herein denotes a solvate in which a solvent molecule is water.

The present invention relates to compounds represented by the following formula (I), geometric isomers and tautomers thereof, or salts, hydrates, or solvates thereof.

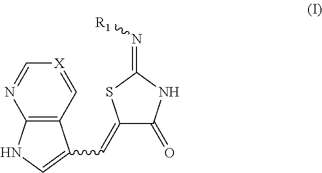

(in the formula, X is CH or N;
R₁ is selected from the group consisting of a straight or branched chain lower alkyl group and a cycloalkyl group that may include a substituent, an aryl group that may have a substituent, an arylalkyl group that may have a substituent, a non-aromatic heterocyclic group that may have a substituent, and a heteroaryl group that may have a substituent, or is a condensed ring group that may have a substituent; and a wavy line, independently for each occurrence, denotes trans (E-form), cis (Z-form) or a mixture (mixed product) thereof.)

Hereinafter, preferable embodiments of the compound in accordance with the present invention are described.

When R₁ is a straight or branched chain lower alkyl group, R₁ can be selected from, for example, a methyl group, an ethyl group, a 1-propyl group, an isopropyl group, an n-butyl group, an s-butyl group, a t-butyl group, an isobutyl group, a 1-pentyl group, a 2-pentyl group, a 3-pentyl group, a 2-methyl-1-butyl group, a 3-methyl-1-butyl group, a 2-methyl-2-butyl group, a 3-methyl-2-butyl group, a 2,2-dimethyl-1-propyl group, a 1-hexyl group, a 2-hexyl group, a 3-hexyl group, a 2-methyl-1-pentyl group, a 3-methyl-1-pentyl group, a 4-methyl-1-pentyl group, a 2-methyl-2-pentyl group, a 3-methyl-2-pentyl group, a 4-methyl-2-pentyl group, a 2-methyl-3-pentyl group, a 3-methyl-3-pentyl group, a 2,3-dimethyl-1-butyl group, a 3,3-dimethyl-1-butyl group, a 2,2-dimethyl-1-butyl group, a 2-ethyl-1-butyl group, a 3,3-dimethyl-2-butyl group, and a 2,3-dimethyl-2-butyl group. Among them, an n-butyl group is preferable.

When R₁ is a cycloalkyl group that may have a substituent, the cycloalkyl group can be selected from, for example, a cyclopropyl group, a cyclobutyl group, a cyclopentyl group, a cyclohexyl group, a cycloheptyl group, and a cyclooctyl group.

Furthermore, the substituent of the cycloalkyl group can be selected from a lower alkyl group, a lower alkoxy group, halogen, a hydrogen group, or the like.

When R₁ is an arylalkyl group that may have a substituent, the arylalkyl group can be selected from, for example, a benzyl group, a phenethyl group, and a 3-phenyl-1-propyl group. Among them, a benzyl group is preferable.

Furthermore, the substituent of the arylalkyl group can include a plurality of substituents in an aryl moiety or an alkyl moiety, or both of the aryl moiety and the alkyl moiety.

The substituents in the aryl moiety can be selected from, for example, fluorine, chlorine, bromine, iodine, a methyl group, an ethyl group, a 1-propyl group, an isopropyl group, an n-butyl group, an s-butyl group, a t-butyl group, an isobutyl group, a 1-pentyl group, a 2-pentyl group, a 3-pentyl group, a 2-methyl-1-butyl group, a 3-methyl-1-butyl group, a 2-methyl-2-butyl group, a 3-methyl-2-butyl group, a 2,2-dimethyl-1-propyl group, a 1-hexyl group, a 2-hexyl group, a 3-hexyl group, a 2-methyl-1-pentyl group, a 3-methyl-1-pentyl group, a 4-methyl-1-pentyl group, a 2-methyl-2-pentyl group, a 3-methyl-2-pentyl group, a 4-methyl-2-pentyl group, a 2-methyl-3-pentyl group, a 3-methyl-3-pentyl group, a 2,3-dimethyl-1-butyl group, a 3,3-dimethyl-1-butyl group, a 2,2-dimethyl-1-butyl group, a 2-ethyl-1-butyl group, a 3,3-dimethyl-2-butyl group, a 2,3-dimethyl-2-butyl group, a trifluoromethyl group, a trichloromethyl group, a difluoromethyl group, a dichloromethyl group, a dibromomethyl group, a fluoromethyl group, a 2,2,2-trifluoroethyl group, a 2,2,2-trichloroethyl group, 2-bromoethyl group, a 2-chloroethyl group, a 2-fluoroethyl group, a 2-iodoethyl group, a pentafluoroethyl group, a 3-chloropropyl group, a 4-fluorobutyl group, a 6-iodohexyl group, 2,2-dibromoethyl group, a methoxy group, an ethoxy group, a 1-propyloxy group, 2-propyloxy group, 2-methyl-1-propyloxy group, a 2-methyl-2-propyloxy group, a 1-butyloxy group, a 2-butyloxy group, 1-pentyloxy group, a 2-pentyloxy group, a 3-pentyloxy group, a 2-methyl-1-butyloxy group, a 3-methyl-1-butyloxy group, a 2-methyl-2-butyloxy group, 3-methyl-2-butyloxy group, a 2,2-dimethyl-1-propyloxy group, a 1-hexyloxy group, a 2-hexyloxy group, a 3-hexyloxy group, a 2-methyl-1-pentyloxy group, a 3-methyl-1-pentyloxy group, a 4-methyl-1-pentyloxy group, a 2-methyl-2-pentyloxy group, a 3-methyl-2-pentyloxy group, a 4-methyl-2-pentyloxy group, a 2-methyl-3-pentyloxy group, a 3-methyl-3-pentyloxy group, a 2,3-dimethyl-1-butyloxy group, a 3,3-dimethyl-1-butyloxy group, a 2,2-dimethyl-1-butyloxy group, a 2-ethyl-1-butyloxy group, a 3,3-dimethyl-2-butyloxy group, and a 2,3-dimethyl-2-butyloxy group. Among them, chlorine, fluorine, bromine, a trifluoromethyl group, or a methoxy group is preferable.

Substituents of the alkyl moiety can be selected from, for example, a methyl group, an ethyl group, a 1-propyl group, an isopropyl group, an n-butyl group, an s-butyl group, a t-butyl group, an isobutyl group, a 1-pentyl group, a 2-pentyl group, a 3-pentyl group, a 2-methyl-1-butyl group, a 3-methyl-1-butyl group, a 2-methyl-2-butyl group, a 3-methyl-2-butyl group, a 2,2-dimethyl-1-propyl group, a 1-hexyl group, a 2-hexyl group, 3-hexyl group, a 2-methyl-1-pentyl group, a 3-methyl-1-pentyl group, a 4-methyl-1-pentyl group, a 2-methyl-2-pentyl group, a 3-methyl-2-pentyl group, a 4-methyl-2-pentyl group, a 2-methyl-3-pentyl group, a 3-methyl-3-pentyl group, a 2,3-dimethyl-1-butyl group, a 3,3-dimethyl-1-butyl group, a 2,2-dimethyl-1-butyl group, a 2-ethyl-1-butyl group, a 3,3-dimethyl-2-buthyl group, and a 2,3-dimethyl-2-butyl group. Among them, a methyl group is preferable.

When R₁ is a non-aromatic heterocyclic group that may have a substituent, the non-aromatic heterocyclic group can be selected from, for example, pyrrolidinyl, dihydropyrrolyl, imidazolidinyl, pyrazolidinyl, oxazolidinyl, thiazolidinyl, piperidyl, dihydropyridinyl, tetrahydropyridinyl, dihydropyrimidinyl, tetrahydropyrimidinyl, hexahydropyrimidinyl, 1,3-oxadinyl, pyranyl, dihydropyranyl, tetrahydropyranyl, tetrahydrofuranyl, dihydrofuranyl, piperazinyl, morpholinyl, thiomorpholinyl, and 1,3-dioxolanyl.

Furthermore, the substituents of the non-aromatic heterocyclic group can be selected from a lower alkyl group, a cycloalkyl group, a lower alkoxy group, halogen, a hydroxyl group, or the like.

When R₁ is an aryl group or a heteroaryl group which may have a substituent, the aryl group can be selected from, for example, a phenyl group, and a naphthyl group. Among them, a phenyl group is preferable. Furthermore, the heteroaryl group can be selected from, for example, a pyridyl group, a thiophenyl group, a furanyl group, a pyrrolyl group, an oxazolyl group, an isoxazolyl group, a thiazolyl group, a thiadiazolyl group, an isothiazolyl group, an imidazolyl group, a triazolyl group, a pyrazolyl group, a furazanyl group, a thiadiazolyl group, an oxadiazolyl group, a pyridazinyl group, a pyrimidinyl group, a pyrazinyl group, a triazinyl group, an indolyl group, an isoindolyl group, an indazolyl group, a chromenyl group, a quinolyl group, an isoquinolyl group, a cinnolinyl group, a quinazolinyl group, a naphthyridinyl group, a phthalazinyl group, a purinyl group, a pteridinyl group, a thienofuranyl group, an imidazo thiazolyl group, a benzofuranyl group, a benzothio phenyl group, a benzooxazolyl group, a benzothiazolyl group, a benzothiadiazolyl group, a benzimidazolyl group, a benzotriazolyl group, an imidazopyridinyl group, a pyrrolopyridinyl group, and a pyrrolopyrimidinyl group. Among them, an indolyl group (for example, an indole-5-yl group, an indole-6-yl group, and an indole-7-yl group), an indazolyl group (for example, a 1H-indazole-6-yl group), a quinolyl group (for example, a quinolin-6-yl group), a benzimidazolyl group (for example, a benzimidazole-2-yl group, a benzimidazole-5-yl group), or a benzotriazolyl group (for example, a benzotriazole-5-yl group) is preferable.

Substituents of the aryl group or the heteroaryl group which may have a substituent can be independently selected from, for example, one to three groups from the following group B.

### Group B:

a straight or branched chain lower alkyl group which may be substituted with a group selected from the group consisting of one to three halogen atoms, a hydroxyl group, an amino group substituted with one or two lower alkyl groups and a non-aromatic heterocyclic group;

a lower alkoxy group;

a hydroxyl group;

a halogen group;

a nitro group;

an amino group that may be substituted with one or two lower alkyl groups;

a lower alkylcarbonylamino group;

a group represented by a formula: —(CH₂)ₖCOOH (wherein k is 0 to 2);

a group represented by a formula: —O—R₂—R₃, wherein R₂ is a single bond, a lower alkylene group, or a cycloalkylene group, or a non-aromatic heterocyclic group that may be substituted with a lower alkyl group and R₃ is a group selected from a hydroxyl group; a carboxyl group; a lower alkoxy group; a lower alkoxycarbonyl group; two lower alkyl groups, or an amino group substituted with one lower alkyl group and one lower alkoxy carbonyl group; and a non-aromatic heterocyclic group that may be substituted with a lower alkyl group; and

a group represented by a formula: —CON(R₄)[(CH₂)ₘ—R₅], wherein m is 0 to 2, R₄ is a hydrogen atom or a lower alkyl group, and R₅ is an amino group substituted with one or two lower alkyl groups.

When the substituent of the aryl group or the heteroaryl group which may have a substituent is a straight or branched chain lower alkyl group that may be substituted with a group selected from the group consisting of one to three halogen atoms, a hydroxyl group, an amino group substituted with one or two lower alkyl groups and a non-aromatic heterocyclic group, such a substituent can be selected from, for example, a methyl group, an ethyl group, a 1-propyl group, an isopropyl group, an n-butyl group, an s-butyl group, a t-butyl group, an isobutyl group, a 1-pentyl group, a 2-pentyl group, a 3-pentyl group, a 2-methyl-1-butyl group, a 3-methyl-1-butyl group, a 2-methyl-2-butyl group, a 3-methyl-2-butyl group, a 2,2-dimethyl-1-propyl group, a 1-hexyl group, a 2-hexyl group, a 3-hexyl group, a 2-methyl-1-pentyl group, a 3-methyl-1-pentyl group, a 4-methyl-1-pentyl group, a 2-methyl-2-pentyl group, a 3-methyl-2-pentyl group, a 4-methyl-2-pentyl group, a 2-methyl-3-pentyl group, a 3-methyl-3-pentyl group, a 2,3-dimethyl-1-butyl group, a 3,3-dimethyl-1-butyl group, a 2,2-dimethyl-1-butyl group, a 2-ethyl-1-butyl group, a 3,3-dimethyl-2-butyl group, a 2,3-dimethyl-2-butyl group, a trifluoromethyl group, a trichloromethyl group, a difluoromethyl group, a dichloromethyl group, a dibromomethyl group, a fluoromethyl group, a 2,2,2-trifluoroethyl group, a 2,2,2-trichloroethyl group, a 2-bromoethyl group, a 2-chloroethyl group, a 2-fluoroethyl group, 2-iodoethyl group, a pentafluoroethyl group, a 3-chloropropyl group, a 4-fluorobutyl group, a 6-iodohexyl group, a 2,2-dibromoethyl group, a methylamino methyl group, a 1-(methylamino)ethyl group, a 2-(methylamino)ethyl group, a 1-(methylamino)propyl group, a 2-(methylamino)propyl group, a 3-(methylamino)propyl group, a dimethylamino methyl group, a 1-(dimethylamino)ethyl group, a 2-(dimethylamino)ethyl group, a 1-(dimethylamino)propyl group, a 2-(dimethylamino)propyl group, a 3-(dimethylamino)propyl group, an ethylamino methyl group, a 1-(ethylamino)ethyl group, a 2-(ethylamino)ethyl group, a 1-(ethylamino)propyl group, a 2-(ethylamino)propyl group, a 3-(ethylamino)propyl group, a diethylamino methyl group, a 1-(diethylamino)ethyl group, a 2-(diethylamino)ethyl group, a 1-(diethylamino)propyl group, a 2-(diethylamino)propyl group, a 3-(diethylamino)propyl group, a hydroxy methyl group, a 2-hydroxy ethyl group, a 1-hydroxy ethyl group, a 3-hydroxy propyl group, a 2-hydroxy propyl group, a 1-hydroxy propyl group, a 4-hydroxy butyl group, a morpholin-4-ylmethyl group, a 1-(morpholin-4-yl)ethyl group, a 2-(morpholin-4-yl)ethyl group, a pyrrolidin-1-ylmethyl group, a 1-(pyrrolidin-1-yl)ethyl group, a 2-(pyrrolidin-1-yl)ethyl group, a piperidin-1-ylmethyl group, a 1-(piperidin-1-yl)ethyl group, a 2-(piperidin-1-yl)ethyl group, or a 3-(piperidin-1-yl)propyl group. Among them, a methyl group, an ethyl group, an isopropyl group, a trifluoromethyl group, a 2-(dimethylamino)ethyl group, a 3-(dimethylamino)propyl group, a 2-(morpholin-4-yl)ethyl group, a 2-(pyrrolidin-1-yl)ethyl group, a 2-(piperidin-1-yl)ethyl group, a 3-(piperidin-1-yl)propyl group, and a hydroxy methyl group are preferable.

When the substituent of the aryl group or the heteroaryl group which may have a substituent is a lower alkoxy group, such a substituent can be selected from, for example, a methoxy group, an ethoxy group, a 1-propyloxy group, a 2-propyloxy group, 2-methyl-1-propyloxy group, a 2-methyl-2-propyloxy group, a 1-butyloxy group, a 2-butyloxy group, 1-pentyloxy group, a 2-pentyloxy group, a 3-pentyloxy group, a 2-methyl-1-butyloxy group, a 3-methyl-1-butyloxy group, a 2-methyl-2-butyloxy group, 3-methyl-2-butyloxy group, a 2,2-dimethyl-1-propyloxy group, a 1-hexyloxy group, a 2-hexyloxy group, a 3-hexyloxy group, a 2-methyl-1-pentyloxy group, a 3-methyl-1-pentyloxy group, a 4-methyl-1-pentyloxy group, a 2-methyl-2-pentyloxy group, a 3-methyl-2-pentyloxy group, a 4-methyl-2-pentyloxy group, a 2-methyl-3-pentyloxy group, a 3-methyl-3-pentyloxy group, a 2,3-dimethyl-1-butyloxy group, a 3,3-dimethyl-1-butyloxy group, a 2,2-dimethyl-1-butyloxy group, a 2-ethyl-1-butyloxy group, a 3,3-dimethyl-2-butyloxy group, and a 2,3-dimethyl-2-butyloxy group. Among them, a methoxy group is preferable.

When the substituent of the aryl group or the heteroaryl group which may have a substituent is halogen, such a substituent is preferably fluorine or chlorine.

When the substituent of the aryl group or the heteroaryl group which may have a substituent is an amino group that may be substituted with one or two lower alkyl groups, such a substituent can be selected from, for example, an amino group, a methylamino group, an ethylamino group, a propylamino group, a dimethylamino group, a diethylamino group, a methylethylamino group, and a methylpropylamino group. Among them, a methylamino group or a dimethylamino group is preferable.

When the substituent of the aryl group or the heteroaryl group which may have a substituent is a lower alkylcarbonylamino group, such a substituent can be selected from, for example, an acetylamino group, a propionylamino group, and a butyrylamino group. Among them, an acetylamino group is preferable.

When the substituent of the aryl group or the heteroaryl group which may have a substituent is a group represented by a formula: —(CH₂)ₖCOOH, wherein k is 0 to 2, such a substituent can be selected from, for example, a carboxyl group (k=0), a carboxy methyl group (k=1), and a carboxyethyl group (k=2). Among them, a carboxyl group or a carboxy methyl group is preferable.

When the substituent of the aryl group or the heteroaryl group which may have a substituent is a group represented by a formula: —O—R₂—R₃, wherein R₂ is a single bond, a lower alkylene group or a cycloalkylene group; R₃ is a group selected from a hydroxyl group; a carboxyl group; a lower alkoxy group; a lower alkoxycarbonyl group; an amino group substituted with two lower alkyl groups or one lower alkyl group and one lower alkoxy carbonyl group; and a non-aromatic heterocyclic group that may be substituted with lower alkyl group, R₂ can be selected from, for example, a single bond, a methylene group, an ethylene group, a trimethylene group, a propylene group, a tetramethylene group, a pentamethylene group, a hexamethylene group, a cyclopropylene group, a cyclobutylene group (for example, 1,1-cyclobutylene, 1,2-cyclobutylene), a cyclopentylene group (for example, 1,1-cyclopentylene, 1,2-cyclopentylene, 1,3-cyclopentylene), and a cyclohexylene group (for example, 1,2-cyclohexylene, 1,3-cyclohexylene, 1,4-cycloxylene). Among them, a single bond, an ethylene group, a trimethylene group or a cyclohexylene group is preferable.

Furthermore, R₃ can be selected from, for example, a hydroxyl group, a carboxyl group, a methoxy group, an ethoxy group, a 1-propyloxy group, a 2-propyloxy group, a 2-methyl-1-propyloxy group, a 2-methyl-2-propyloxy group, a 1-butyloxy group, a 2-butyloxy group, a 1-pentyloxy group, a 2-pentyloxy group, a 3-pentyloxy group, a 2-methyl-1-butyloxy group, a 3-methyl-1-butyloxy group, a 2-methyl-2-butyloxy group, a 3-methyl-2-butyloxy group, a 2,2-dimethyl-1-propyloxy group, a 1-hexyloxy group, a 2-hexyloxy group, a 3-hexyloxy group, a 2-methyl-1-pentyloxy group, a 3-methyl-1-pentyloxy group, a 4-methyl-1-pentyloxy group, a 2-methyl-2-pentyloxy group, a 3-methyl-2-pentyloxy group, a 4-methyl-2-pentyloxy group, a 2-methyl-3-pentyloxy group, a 3-methyl-3-pentyloxy group, a 2,3-dimethyl-1-butyloxy group, a 3,3-dimethyl-1-butyloxy group, a 2,2-dimethyl-1-butyloxy group, a 2-ethyl-1-butyloxy group, a 3,3-dimethyl-2-butyloxy group, 2,3-dimethyl-2-butyloxy group, a methoxycarbonyl group, an ethoxycarbonyl group, a 1-propyloxycarbonyl group, a 2-propyloxycarbonyl group, a dimethylamino group, a diethylamino group, a methylethylamino group, a methylpropylamino group, an N-methoxycarbonyl-N-methylamino group, an N-methoxycarbonyl-N-ethylamino group, an N-ethoxycarbonyl-N-methylamino group, an N-methoxycarbonyl-N-ethylamino group, a pyrrolidinyl group, a dihydropyrrolyl group, an imidazolidinyl group, a pyrazolidinyl group, an oxazolidinyl group, a thiazolidinyl group, a piperidyl group, a dihydropyridinyl group, a tetrahydropyridinyl group, a dihydropyrimidinyl group, a tetrahydropyrimidinyl group, a hexahydropyrimidinyl group, a 1,3-oxadinyl group, a pyranyl group, a dihydropyranyl group, a tetrahydropyranyl group, a tetrahydrofuranyl group, a dihydrofuranyl group, a piperazinyl group, a morpholinyl group, a thiomorpholinyl group, 1,3-dioxolanyl group, a 1-methylpyrrolidin-2-yl group, a 1-methylpyrrolidin-3-yl group, a 1-methylimidazolidin-2-yl group, a 1-methylimidazolidin-3-yl group, a 1-methylpyrazolidin-3-yl group, a 1-methylpyrazolidin-4-yl group, a 1-methylpiperidin-2-yl group, a 1-methylpiperidin-3-yl group, a 1-methylpiperidin-4-yl group, 4-methylpiperazin-1-yl group, a 1-methylpiperazin-4-yl group, a 1-ethylpyrrolidin-2-yl group, a 1-ethylpyrrolidin-3-yl group, a 1-ethylimidazolidin-2-yl group, a 1-ethylimidazolidin-3-yl group, a 1-ethylpyrazolidin-3-yl group, a 1-ethylpyrazolidin-4-yl group, a 1-ethylpiperidin-2-yl group, a 1-ethylpiperidin-3-yl group, 1-ethylpiperidin-4-yl group, a 4-ethylpiperazin-1-yl group, and a 1-ethylpiperazin-4-yl group. Among them, a hydroxyl group, a carboxyl group, a methoxy group, an ethoxy carbonyl group, a dimethyl amino group, a diethyl amino group, an N-methoxycarbonyl-N-methylamino group, a pyrrolidinyl group (for example, pyrrolidin-1-yl group), a piperidinyl group (for example, piperidin-1-yl group), a morpholinyl group (for example, a morpholin-4-yl group), a 1-methylpyrrolidin-3-yl group, a 1-methyl piperidin-4-yl group, or a 4-methyl piperazin-1-yl group is preferable.

When the substituent of the aryl group or the heteroaryl group which may have a substituent is a group represented by a formula: —CON(R₄)[(CH₂)ₘ—R₅], wherein m is 0 to 2, R₄ is a hydrogen atom or a lower alkyl group, and R₅ is an amino group that is substituted with one or two lower alkyl groups, R₄ can be selected from, for example, a hydrogen atom, a methyl group, an ethyl group, 1-propyl group, an isopropyl group, an n-butyl group, an s-butyl group, a t-butyl group, and an isobutyl group. Among them, a hydrogen atom or a methyl group is preferable.

Furthermore, R₅ can be selected from, for example, a methyl amino group, an ethyl amino group, a propyl amino group, a dimethyl amino group, a diethyl amino group, a methylethylamino group, and a methyl propyl amino group. Among them, a dimethyl amino group is preferable.

When R₁ is a condensed ring group of a cycloalkyl group or a non-aromatic heterocyclic group and an aryl group, such a condensed ring group can be selected from, for example, an indanyl group, a 1,2,3,4-tetrahydronaphthyl group, a 3,4-dihydro-2H-1,4-benzooxadinyl group, a 3,4-dihydro-2H-1,4-benzothiazinyl group, a 1,3-benzodioxolyl group, a 2,3-dihydro-1,4-benzodioxinyl group, a chromanyl group, an isochromanyl group, a 3,4-dihydro-2H-1-benzothiopyranyl group, a 3,4-dihydro-1H-2-benzothiopyranyl group, an indolinyl group, an isoindolinyl group, a 1,2,3,4-tetrahydroquinolyl group, and a 1,2,3,4-tetrahydroisoquinolyl group. Among them, an indanyl group (for example, an indan-4-yl group, an indan-1-yl group), or a 1,3-benzodioxolyl group (for example, a 1,3-benzodioxole-5-yl group) is preferable.

Furthermore, the condensed ring group may have a substituent. The substituent can be specifically selected from a lower alkyl group, a cycloalkyl group, a lower alkoxy group, halogen, a hydroxyl group, or the like.

In the formula (I), a wavy line, independently for each occurrence, denotes trans (E-form), cis (Z-form) or a mixture (mixed product) thereof. Each isomer of the wavy line can be specifically represented by the following formulae (I-I) to (I-IV).

The isomers represented by the formulae (I-I) to (I-IV) can be mutually converted into each other in a solvent in the presence of, for example, an acid or a base. In the present specification, the “mixture of cis and trans” or the “mixture of an E-form and a Z-form” means that such states are included. Although depending upon the isolation methods, when isolation is carried out, the isomer can generally have geometric isomerism represented by the formula (I-I).

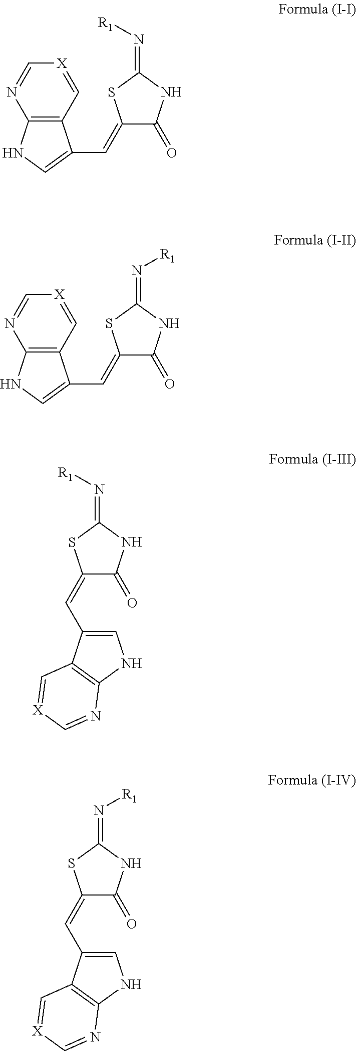

The compound of the present invention may have an isomer, for example, depending upon the types of substituents. The present specification may describe a chemical structure of only one embodiment in such isomers. However, the present invention encompasses all types of isomers (geometric isomer, optical isomer, stereoisomer, tautomer, and the like) having chemical structures that can be generated, and further encompasses one separated from the isomer, or the mixture with the isomer.

Various isomers can also be purified and isolated by using usual separation methods, for example, recrystallization, a diastereomeric salt method, an enzyme fractionation method, various types of chromatographies (for example, thin-layer chromatography, column chromatography, and the like). Alternatively, a mixture of isomers can be employed as long as the intended actions are maintained.

Furthermore, the compound of the present invention may form a salt. Any salts may be encompassed in the present invention as long as they are pharmaceutically acceptable salts.

Specific examples of such salts include an inorganic acid salt, an organic acid salt, an inorganic base salt, an organic base salt, an acidic amino acid salt, and a basic amino acid salt. Among them, examples of the inorganic acid salt include hydrochloride, hydrobromate, sulfate, nitrate, and phosphate. On the other hand, examples of the organic acid salt include acetate, succinate, fumarate, maleate, tartrate, citrate, lactate, stearate, benzoate, methanesulfonate, and p-toluene sulfonate.

Furthermore, the compound of the present invention may be a pharmaceutically acceptable prodrug. The pharmaceutically acceptable prodrug in the present specification is a derivative of the compound of the present invention that has been modified by a group capable of chemically or metabolically degrading the compound of the present invention. The prodrug is a derivative that is demodified after it is given to a living body, generates an original compound, and exhibits the original drug effect.

Furthermore, various types of hydrates or solvates of the compounds and pharmaceutically acceptable salts of the present invention may be encompassed in the present invention.

Furthermore, the compounds in accordance with the present invention may have polymorphisms. Substances of such polymorphisms may be encompassed in the present invention.

### Typical Production Process of Compound of the Present Invention

The compound represented by the formula (I) in accordance with the present invention can be produced by applying various known organic synthesis reactions. At this time, depending upon the types of functional groups, the functional group may be substituted with an appropriate protecting group in the stage of raw materials or intermediate products. As selection, and introduction and deprotection of such a protecting group, for example, the method described in “Greene's Protective Groups in Organic Synthesis (the 4th edition, Wiley-Interscience, 2007)” can be employed. In production of the compounds of the present invention, commercially available compounds, or compounds produced in usual methods can be used for compounds of raw materials.

The typical production process is shown below.

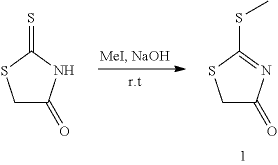

2-Thioxothiazolidin-4-one is reacted with methyl iodide to produce compound 1. The reaction is carried out at 18-33° C., normally 25° C., stirred for 16 hours-overnight, in the presence of a base such as diisopropylethylamine, sodium hydroxide. As the reaction solvent, water, methanol, ethanol or DMF is preferably used.

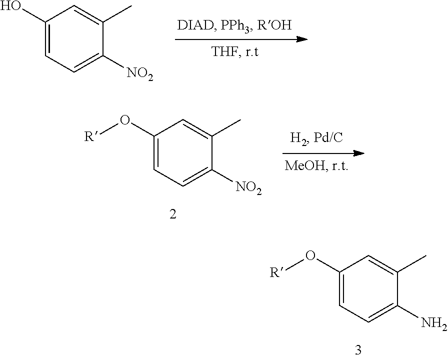

Compound 2 is synthesized by reacting 3-methyl-4-nitrophenol with R′OH in the presence of Ph₃P, and diisopropyl azodicarboxylate or diethyl azodicarboxylate. The reaction is carried out by stirring at 18-28° C. for 8-18 hours. As the reaction solvent, dichloromethane, tetrahydrofuran, 1,4-dioxane, diethylether, or toluene is preferably used.

Compound 3 is obtained by reducing compound 2 using the catalytic reduction method. The reaction is carried out by stirring at 18-33° C., normally 25° C. for 3-12 hours under H₂ atmosphere. As the reaction solvent, methanol, ethanol, dichloromethane, ethyl acetate or these mixed solvent is preferably used.

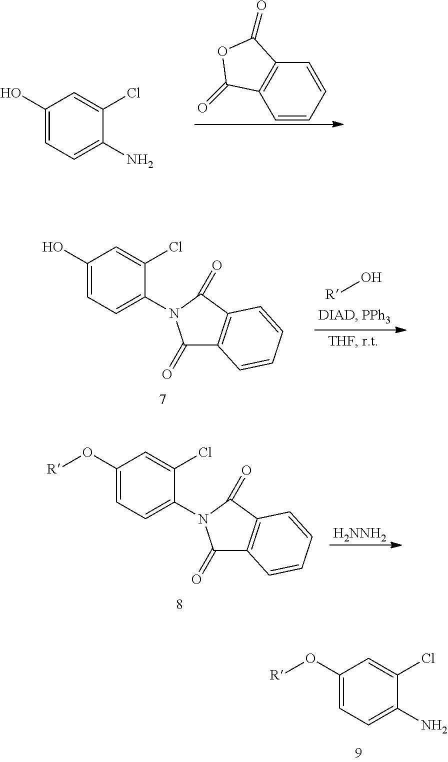

Compound 7 is synthesized by reacting 4-amino-3-chlorophenol hydrochloride with 1,2-benzenedicarboxylic anhydride. The reaction is carried out for 1-10 hours at 120-140° C. in the presence of a solvent such as ethyl acetate, dimethylformamide, N-methylpyrrolidone, pyridine or dimethylacetamide.

Compound 8 is synthesized by reacting compound 7 with R′OH in the presence of Ph₃P and diisopropyl azodicarboxylate or diethyl azodicarboxylate. The reaction is carried out by stirring at 18-28° C. for 8-18 hours. As the reaction solvent, dichloromethane, tetrahydrofuran, 1,4-dioxane, diethylether or toluene is preferably used.

Compound 9 is obtained by treating compound 8 with acid or base in the presence or absence of the solvent. As acid, hydrobromide or hydrochloride is preferably used. As the base, hydrazine hydrate or butylamine is preferably used. The reaction is carried out by stirring for 1 hour-4 days, at 80-100° C. As the reaction solvent, aqueous methanol, aqueous ethanol, acetone, or a mixed solvent of acetone and ethanol is preferably used.

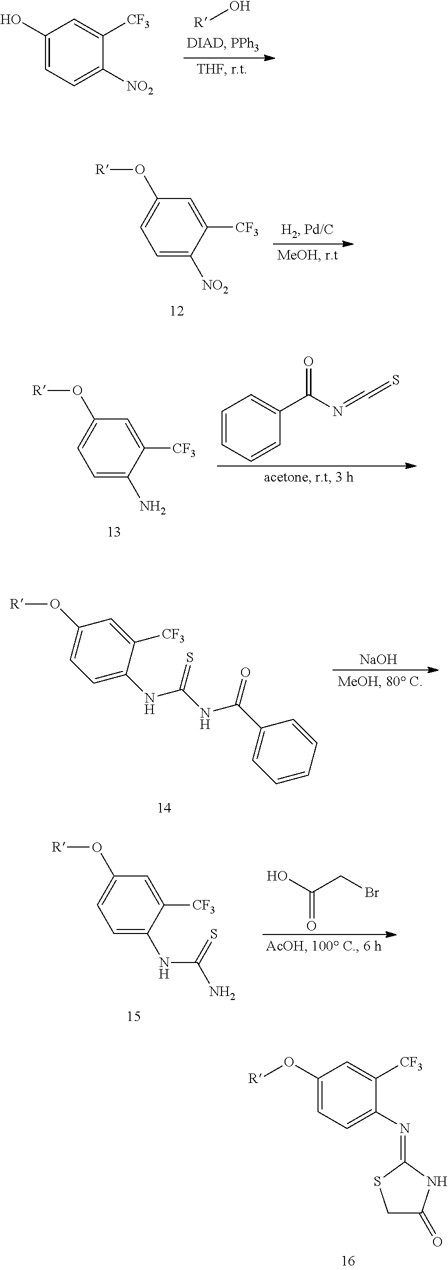

Compound 12 is synthesized by reacting 4-nitro-3-trifluoromethyl-phenol with ROH in the presence of Ph₃P and diisopropyl azodicarboxylate or diethyl azodicarboxylate. The reaction is carried out by stirring at 18-28° C. for 8-18 hours. As the reaction solvent, dichloromethane, tetrahydrofuran, 1,4-dioxane, diethylether or toluene is preferably used.

Compound 13 is obtained by reducing compound 12 using the catalytic reduction method. The reaction is carried out by stirring at 18-33° C., normally 25° C., for 3-12 hours under H₂ atmosphere. As the reaction solvent, methanol, ethanol, dichloromethane, ethyl acetate or these partially mixed solvent is preferably used.

Compound 14 is synthesized by reacting compound 13 with benzoyl isothiocyanate. The reaction is carried out for 30 min-6 hours at 18-33° C. in the presence of a solvent such as acetone, 1,4-dioxane, ethanol, chloroform, tetrahydrofuran, dichloromethane or acetonitrile.

Compound 15 is obtained by the hydrolysis of compound 14 in the presence of a base such as sodium hydroxide or lithium hydroxide. The reaction is carried out by stirring at 60-80° C. for 2-4 hours. As the reaction solvent, water, methanol, ethanol, tetrahydrofuran or these partially mixed solvent is preferably used.

Compound 16 is synthesized by reacting compound 15 with a reactant such as methyl bromoacetate, ethyl bromoacetate, methyl chloroacetate or ethyl chloroacetate in the presence of an acid such as acetic acid. The reaction is carried out by stirring at 45-80° C. for 20 min-10 hours. As the reaction solvent, ethanol, acetone, dimethylformamide or 1,4-dioxane is preferably used.

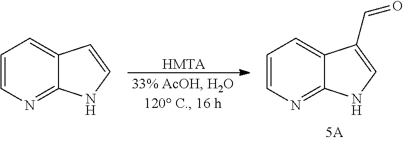

Compound 5A is synthesized by reacting 1H-Pyrrolo[2,3-b]pyridine with hexamethylenetetramine in the presence of acetic acid or phosphoryl chloride. The reaction is carried out by stirring at room temperature-120° C. for 12-16 hours. As the reaction solvent, water or dimethylformamide is preferably used.

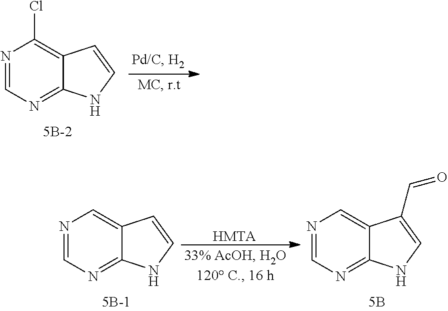

Compound 5B-1 is obtained by reducing compound 5B-2 using the catalytic reduction method. The reaction is carried out by stirring at 18-33° C., normally 25° C., for 3-12 hours under H₂ atmosphere. As the reaction solvent, dichloromethane, methanol, ethanol, dichloromethane, ethyl acetate or these partially mixed solvent is preferably used.

Compound 5B is synthesized by reacting compound 5B-1 with hexamethylenetetramine in the presence of acetic acid or phosphoryl chloride. The reaction is carried out by stirring at room temperature-120° C. for 12-16 hours. As the reaction solvent, water or dimethylformamide is preferably used.

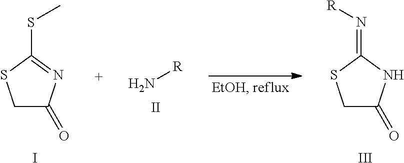

wherein R—NH₂ is

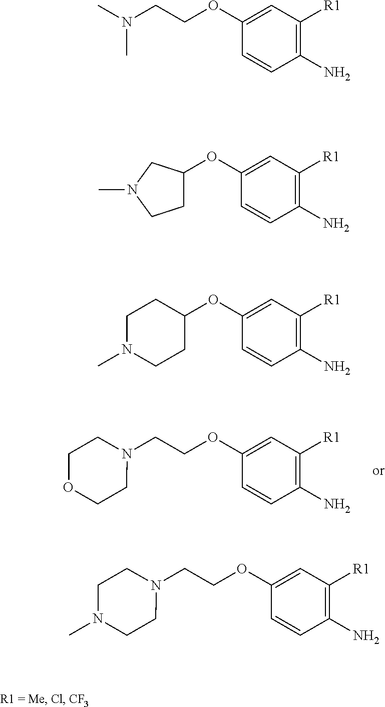

The compound of formula I is reacted with the compound of formula II to produce the compound of formula III. The reaction is carried out at the range of 0° C. to reflux temperature by stirring during 10 minutes to 24 hours, using either the same or excess amount of the compound of formula I and the compound of formula II. As the reaction solvent, a protic polar solvent such as water, methanol, ethanol, or propanol, is preferably used.

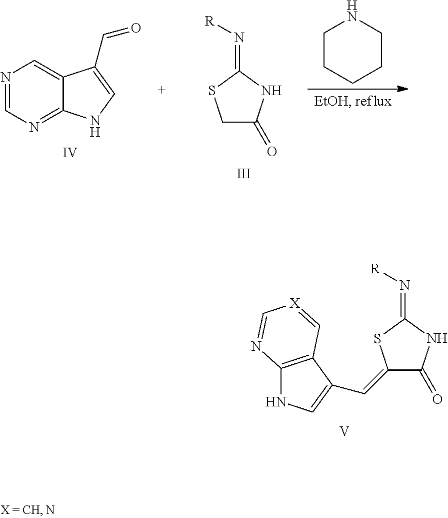

wherein X is a CH or a nitrogen atom, and R—NH₂ is

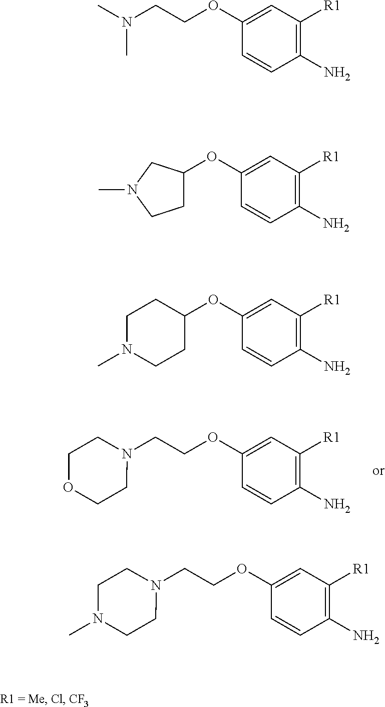

The compound of formula IV is reacted with the compound of formula III to produce the compound of formula V. The reaction is carried out at the range of 80° C. to reflux temperature by stirring during 8 hours to 24 hours, using either the same or excess amount of the compound of formula IV and the compound of formula III, in the presence of a base, such as piperidine or sodium acetate. As the reaction solvent, a polar solvent such as ethanol or acetic acid is preferably used.

The compounds of the present invention can be isolated or purified by employing usual chemical operations such as extraction, concentration, distillation, crystallization, filtration, recrystallization, various chromatographies, and the like.

Furthermore, the present invention provides a pharmaceutical composition including the compound and pharmaceutically acceptable carriers in accordance with the present invention.

When the compounds or the salts, hydrates, or solvates thereof in accordance with the present invention are administered, the administration form is not particularly limited. They may be administered by oral administration or parenteral administration in conventional methods. They can be formulated and administered in dosage forms of, for example, tablets, powder, granules, capsules, syrup, troches, inhalant, suppositories, injection, ointment, eye ointment, eye drop, nasal drop, ear drop, cataplasm, lotion, and the like.

In preparation, commonly used excipient, binder, lubricant, colorant, flavoring agent, as well as stabilizer, emulsifying agent, absorption promoter, surfactant, pH regulating agents, antiseptics, anti-oxidant, and the like, can be used as necessary, and preparation is achieved by usual methods while blending components that are generally used as raw materials of pharmaceutical formulation.

For example, in production of oral formulations, an excipient, and further, as necessary, a binder, a disintegrator, a lubricant, colorant, a flavoring agent, and the like, are mixed to the compounds or the salts, hydrates, or solvates thereof in accordance with the present invention, and the mixture is formed into powder, fine granules, granules, tablets, coating tablets, capsules, and the like, by usual methods.

For example, in production of liquid agents such as syrup and injection formulations, pH regulating agents, resolvents, tonicity agents, and the like, optionally together with dissolution aids, stabilizers, and the like, are mixed to the compound or the salts, hydrates, or solvates thereof in accordance with the present invention, and then the mixture is formed into preparations by usual methods.

The dosage amount of the compounds in accordance with the present invention can be appropriately selected depending on the severity of symptom, age, sex, body weight, dosage form, type of the salt, particular type of diseases, and the like.

In the case of oral administration, the compounds are administered appropriately to an adult in a dose of about 10 mg to 2000 mg per day, and preferably 50 mg to 1000 mg per day. The compounds are administered once to several times a day.

In the case of intravenous administration, the compounds are administered appropriately to an adult in a dose of about 1 mg to 1000 mg per day, and preferably from 10 mg to 100 mg. The compounds are administered once to several times a day.

### EXAMPLES

For example, the compound of the present invention can be produced by processes described in the following Examples. However, the Examples are just illustrative, and the compounds of the present invention are not limited to the compounds described in the below mentioned Examples.

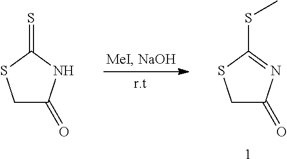

### Synthesis of Compound 1

2-thioxothiazolidin-4-one (25 g, 188 mmol) were dissolved in aqueous NaOH (2%, 375 mL) at room temperature. To this solution methyl iodide (29.5 g, 206.8 mmol) was added, and the reaction mixture was stirred overnight at room temperature. The reaction mixture was diluted with dichloromethane (DCM), washed with cold saturated aqueous NaHCO₃ and water, and dried over anhydrous Na₂SO₄. The DCM was evaporated till dryness and the residue was crystallized from methanol to obtain compound 1 as pure product (Yield: 38˜51%).

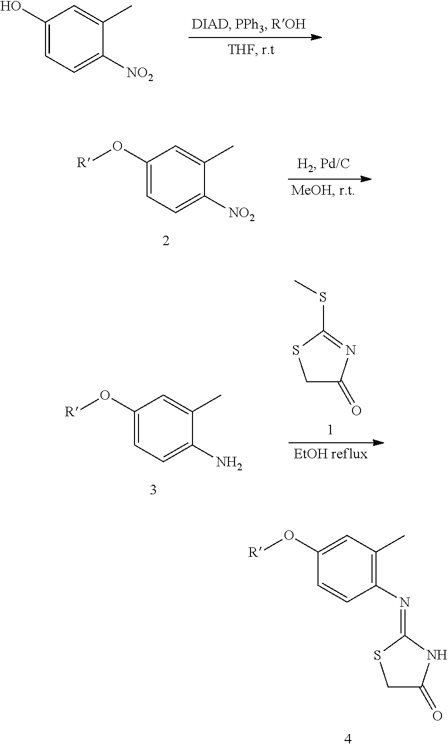

### Synthesis of Compound 2

3-methyl-4-nitrophenol (1.53 g, 10 mmol) was dissolved in DCM (100 mL) and triphenylphosphin (Ph₃P) (13.10 g, 50 mmol) was added into the solution. The mixture was stirred at room temperature for 30 min. Then alcohol R′OH (13 mmol) was added into the mixture and stirred at room temperature for 5 min. Then diisopropyl azodicarboxylate (10.10 g, 50 mmol) dissolved in 20 ml of DCM was dropped into the mixture and the mixture was stirred at room temperature for 5 hours. The solvent was evaporated in vacuo to dry and the residue was dissolved in 4 N HCl, washed with ethylacetate (EA). The aqueous layer was basified with KOH aq., extracted with EA, washed with water and brine, dried over MgSO₄, then concentrated to afford the desired product, compound 2 (Yield: 41˜86%). This product was used directly in the next step.

### Synthesis of Compound 3

Compound 2 (5.36 mmol) was dissolved in methanol (MeOH) (25 mL) then Pd/C (120 mg) was added. The mixture was stirred under H₂ overnight, filtered and the filtrate was evaporated in vacuo to afford the desired product, compound 3 which was used directly in the next step (98˜99% yield).

### Synthesis of Compound 4

Compound 3 (5.26 mmol) and compound 1 (0.773 g, 5.26 mmol) were dissolved in ethanol (EtOH) (30 mL) and the mixture was stirred at reflux overnight and the solvent was evaporated in vacuo to afford the crude product which was purified with silica gel chromatography (DCM/MeOH=10/1) to afford the pure product, compound 4 (13˜66% yield).

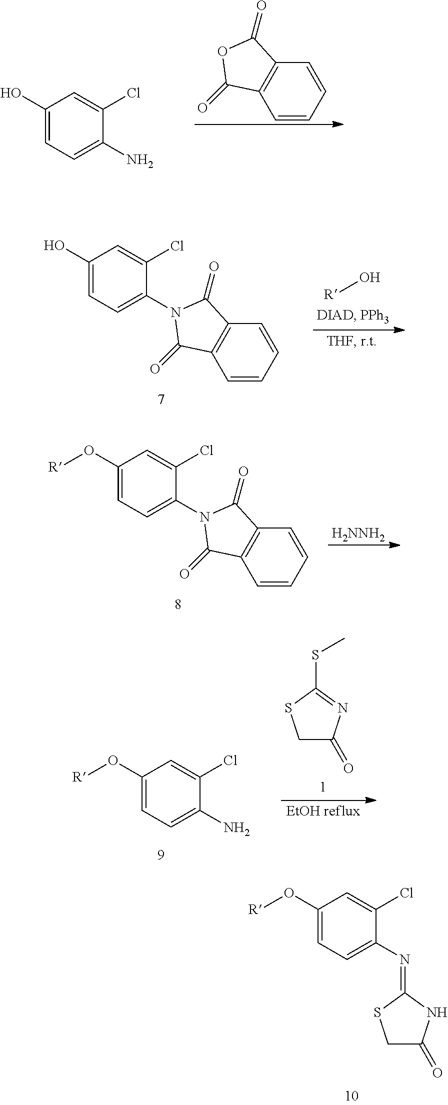

### Synthesis of Compound 7

4-amino-3-chlorophenol hydrochloride (1.8 g, 10 mmol) and 1,2-benzenedicarboxylic anhydride (1.48 g, 10 mmol) in acetic acid (AcOH) (40 mL) were stirred at 120° C. overnight. The mixture was concentrated to afford the crude product which was dissolved in EA and washed with water and brine, dried over Na₂SO₄, filtered and the filtrate was evaporated in vacuum to afford the desired product, compound 7 (Yield: 73.1˜93.7%).

### Synthesis of Compound 8

Compound 7 (34.8 mmol) and PPh₃ (27 g, 104.4 mmol) was added into DCM (300 mL). The mixture was stirred at room temperature for 30 min. Then alcohol R′OH (41.8 mmol) in DCM (20 mL) was added into the solution, and the mixture was stirred at room temperature for 10 min. Then DIAD (21 g, 104.4 mmol) in DCM (20 mL) was added into the solution. The mixture was stirred at room temperature for 5 hours. The mixture was concentrated in vacuo to dryness which was dissolved in 2 N HCl (aq) and washed with EA. The aqueous layer was basified with NaOH solution, extracted with EA, washed with water and brine, dried over Na₂SO₄, filtered and the filtrate was evaporated in vacuo to afford the desired product, compound 8 (Yield: 44.35˜51.8%).

### Synthesis of Compound 9

Compound 8 (15.4 mmol) was heated under reflux with hydrazine hydrate (6 mL) in EtOH (200 mL) for 4 hours. The mixture was filtered and the filtrate concentrated to afford the crude product which was dissolved in EA and washed with water and brine, dried over Na₂SO₄, filtered and the filtrate was evaporated in vacuo to afford the desired product, compound 9 (Yield: 39.0˜91.3%).

### Synthesis of Compound 10

Compound 9 (12.1 mmol) and 1 (1.96 g, 13.3 mmol) in EtOH (50 mL) were stirred at 80° C. for 10 hours. The mixture was concentrated in vacuo to dryness which was purified with silica gel chromatography (DCM/MeOH=20:1) to afford the desired product, compound 10 (Yield: 16.5˜24%).

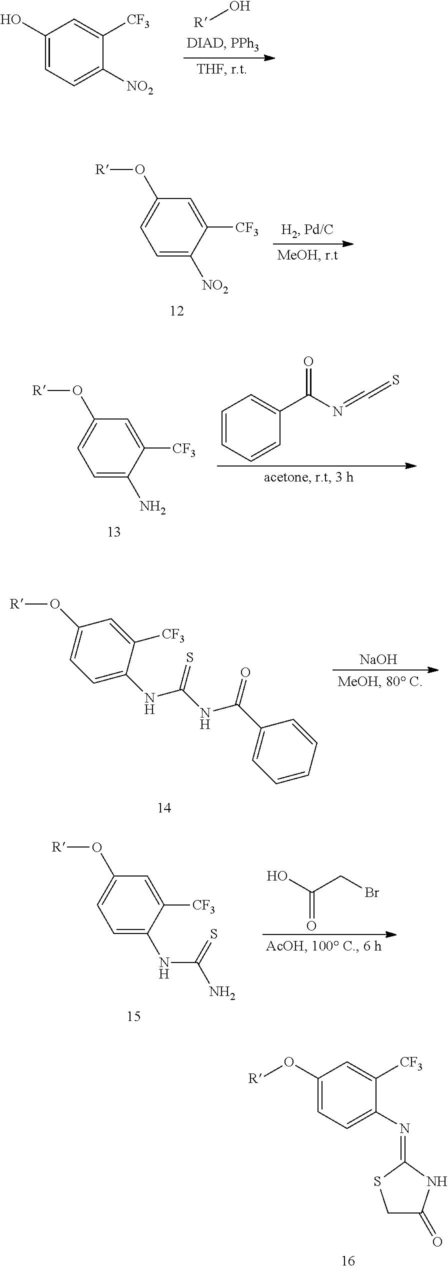

### Synthesis of Compound 12

4-nitro-3-trifluoromethyl-phenol (17 mmol) was dissolved in DCM (150 mL) and PPh₃ (13.5 g, 52.2 mmol) was added into the solution. The mixture was stirred at room temperature for 30 min. Then alcohol R′OH (17 mmol) in DCM (10 mL) was added into the solution, and the mixture was stirred at room temperature for 10 min. Then DIAD (10.5 g, 52.2 mmol) in DCM (10 mL) was added into the solution. The mixture was stirred at room temperature for overnight. The mixture was concentrated in vacuo to dryness which was dissolved in 2 N HCl (aq) and washed with EA. The aqueous layer was basified with NaOH solution, extracted with EA, washed with water and brine, dried over Na₂SO₄, and filtered and the filtrate was evaporated in vacuo to afford the desired product, compound 12 (Yield: 53.6%).

### Synthesis of Compound 13

Compound 12 (13.6 mmol) was dissolved in MeOH (250 mL) then Pd/C (385 mg) was added. The mixture was stirred under H₂ for overnight. The reaction solution was filtered, and the filtrate was concentrated under a reduced pressure to remove the solvent to obtain the desired oil product (compound 13), which was directly used in the next step (crude yield: 96%).

### Synthesis of Compound 14

The solution of compound 13 (1 mmol) and benzoyl isothiocyanate (163 mg, 1 mmol) in 5 mL of acetone was stirred at room temperature for 1 hour. The reaction mixture was concentrated to remove acetone and obtain the crude product (compound 14), which was directly used in the next step (crude yield: 90%).

### Synthesis of Compound 15

NaOH solution (1 M, 1.1 mmol, 1.2 eq) was added to a stirred mixture of compound 14 (0.9 mmol) in 8 mL of EtOH. The reaction mixture was refluxed for 3 hours, cooled and concentrated. The white solid was treated with water (20 mL), extracted with ethyl acetate, the organic layer was concentrated to obtain the crude product, which was directly used in the next step. (crude yield: 72.7˜77.5%).

### Synthesis of Compound 16

Compound 15 (0.65 mmol) was dissolved in 10 mL of ethanol. To this solution, BrCH₂COOMe (129 mg, 0.85 mmol) was added, and the mixture was stirred at 50° C. for 2 hours. After cooling to room temperature, the mixture was neutralized with aqueous ammonia. The mixture was concentrated to obtain the white solid. It was dissolved in EA and washed with water. The organic layer was concentrated to obtain the crude product. The crude product was purified by silica gel chromatography (DCM: MeOH=10:1) to obtain desired compound 16 (yield 22.7˜48.3%).

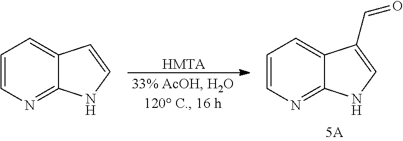

### Synthesis of Compound 5A

To 1H-Pyrrolo[2,3-b]pyridine (5.0 g, 42.4 mmol) in water (35 mL), were added hexamethylenetetramine (8.327 g, 59.4 mmol), and acetic acid (17.4.0 mL, 296.8 mmol). The reaction mixture was refluxed for 12 hours. Water (120 mL) was added and the reaction was cooled to room temperature. The reaction mixture was filtrated, washed with water, and dried to give compound 5A (2.40 g, 39% yield).

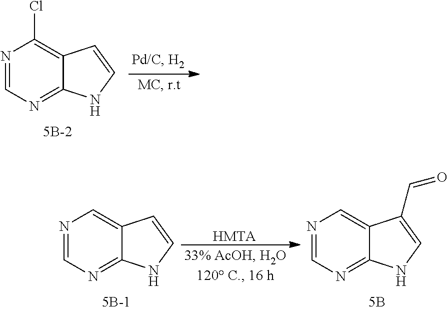

### Synthesis of Compound 5B-1

Compound 5B-2 (5 g, 32.6 mmol) was dissolved in MeOH (100 mL), 10% Pd/C was added. The reaction was stirred under H₂ at room temperature for overnight. The reaction was filtered, and concentrated. The crude product (compound 5B-1) was used directly in the next step (3.88 g).

### Synthesis of Compound 5B

To compound 5B-1 (3.88 g, 32.6 mmol) in water (26 mL), were added hexamethylenetetramine (6.4 g, 45.6 mmol), and acetic acid (13 mL, 228.2 mmol). The reaction was refluxed for 12 hours. Na₂CO₃ (aq) was added to adjust pH to 8˜9. The mixture was extracted with ethylacetate (EtOAc) (3×150 ml), dried over Na₂SO₄, concentrated to afford compound 5B (0.68 g, white powder, 22%).

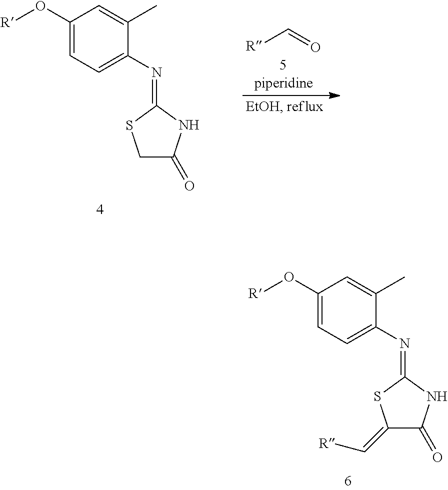

### Synthesis of Compound 6

Compound 4 (0.5 mmol), piperidine (0.07 ml) and compound 5A or 5B (0.5 mmol) were dissolved in EtOH (5 mL) and the mixture was stirred at reflux for 7 hours and the solvent was evaporated in vacuo to afford the crude product which was purified with silica gel chromatography (DCM/MeOH=10/1) to afford the desired product 6 (31˜60.0% yield).

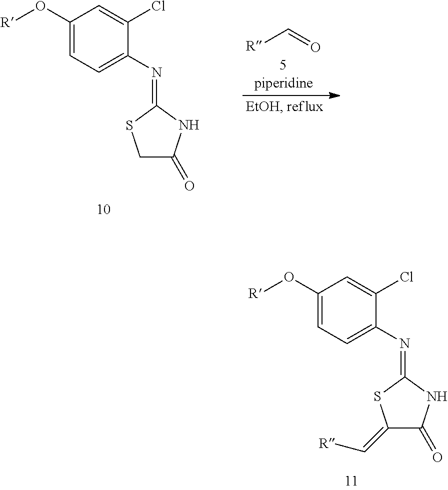

### Synthesis of Compound 11

Compound 10 (0.5 mmol) was dissolved in EtOH (7 ml), Compound 5A or 5B (0.5 mmol) and piperidine (0.1 ml) was added. The reaction mixture was refluxed overnight. The reaction mixture was cooled to room temperature, and concentrated. The crude product was purified by silica gel chromatography (DCM/MeOH=10:1) to afford compound 11 (yield: 33˜36%).

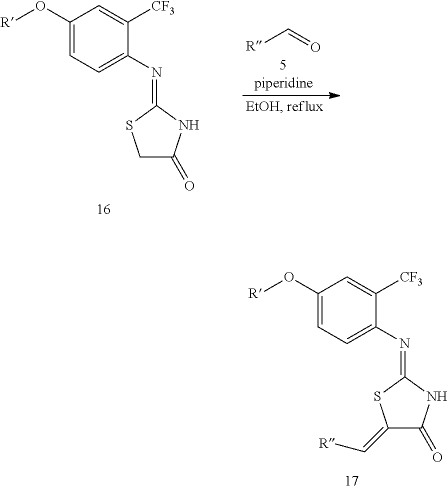

### Synthesis of Compound 17

Compound 16 (0.35 mmol) was dissolved in EtOH (4 ml), Compound 5 (0.35 mmol) and piperidine (0.1 ml) was added. The reaction mixture was refluxed overnight. The reaction mixture was cooled to room temperature, evaporated in vacuo to afford the crude product which was purified with silica gel chromatography (DCM/MeOH=10:1) to afford compound 17 (yield: 51˜55%).

Compounds of Examples shown in the following Tables were produced by using the corresponding raw materials by the above-mentioned production processes or production processes similar thereto. Table 2 shows the structure, the name, and the physicochemical data of each compound of Example.

HPLC of each compound described in Table 2 was conducted in the following conditions.

TABLE 1
Method A

| | |
|---|---|
| Mobile phase | A: water of 0.1% Formic acid |
| | B: ACN |

| | Time | Flow rate | | |
|---|---|---|---|---|
| | (min) | (ml/min) | A % | B % |
| Gradient | 7 | 1 | 100 | 0 |
| | 8.5 | 1 | 100 | 0 |
| | 11 | 1 | 5 | 95 |
| | 12 | 1 | 5 | 95 |

| | |
|---|---|
| Wave length | 254 nm |

TABLE 2
Method B

| | |
|---|---|
| Mobile phase | A: water of 0.1% TFA |
| | B: ACN |

| | Time | Flow rate | | |
|---|---|---|---|---|
| | (min) | (ml/min) | A % | B % |
| Gradient | 2 | 1 | 95 | 5 |
| | 13 | 1 | 5 | 95 |
| | 18 | 1 | 5 | 95 |
| | 19 | 1 | 95 | 5 |
| | 20 | 1 | 95 | 5 |

| | |
|---|---|
| Wave length | 254 nm |

TABLE 3
C method

| | |
|---|---|
| Column | Waters Atlantis HILIC Silica 5.0 um 4.6 * 150 mm |
| Column Temp | 25° C. |
| Pump | Agilent Quaternary pump G1310A |
| Mobile phase | C: Acetonirile(0.03% TFA) D: H₂O(0.03% TFA) |
| Gradient | 95-60% C in 12 min |
| 0 min | 5% D: H₂O(0.03% TFA), 95% C: |
| | Acetonirile(0.03% TFA) |
| 12 min | 40% D: H₂O(0.03% TFA), 60% C: |
| | Acetonirile(0.03% TFA) |
| Flow rate | 1.0 ml/min |
| Detector | Agilent VWD G1315B |
| Detection | 254 nm |
| wavelength | |
| Autosampler | Agilent G1313A |
| Injection volumn | 1 ul |

TABLE 4

| Compound | | | Dtctd | | |
|---|---|---|---|---|---|
| No | Structure | Name | Mass | 1H NMR | HPLC |
| 1 | 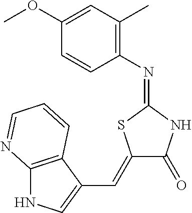 | (2Z,5Z)-2-(4-methoxy-2- methylphenyl)azamethylene- 5-(1H-pyrrolo[2,3-b]pyridin-3- yl)methylene-1,3-thiazolidin- 4-one | 365.1 | ¹H NMR (300 MHz, DMSO-d6) δ = 8.31 (d, J = 5.8, 2H), 7.85 (s, 1H), 7.57 (s, 1H), 7.21 (s, 1H), 6.91-6.88 (m, 2H), 3.76 (s, 3H), 2.14 (s, 3H) | A Method 6.172 |
| 2 | 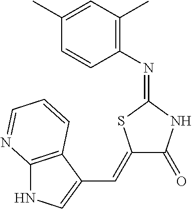 | (2Z,5Z)-2-(2,4- dimethylphenyl)azamethylene- 5-(1H-pyrrolo[2,3-b]pyridin-3- yl)methylene-1,3-thiazolidin- 4-one | 349.1 | ¹H NMR (300 MHz, DMSO-d6) δ = 8.31 (d, J = 5.4 Hz, 2H), 7.85 (s, 1H), 7.56 (s, 1H), 7.22-7.04 (m, 3H), 2.28 (s, 3H), 2.11 (s, 3H) | A Method 6.347 |
| 3 | 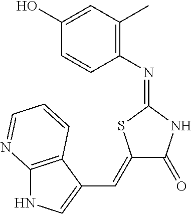 | (2Z,5Z)-2-(4-hydroxy-2- methylphenyl)azamethylene- 5-(1H-pyrrolo[2,3-b]pyridin-3- yl)methylene-1,3-thiazolidin- 4-one | 351.1 | ¹H NMR (300 MHz, DMSO-d6) δ = 12.34 (bs, 1H), 9.31 (bs, 1H), 8.28 (d, J = 7.6 Hz, 2H), 7.81 (s, 1H), 7.56 (s, 1H), 7.17 (t, J = 5.4 Hz, 1H), 6.82 (s, 1H), 6.61 (m, 2H), 2.07 (s, 3H) | A Method 5.794 |
| 4 | 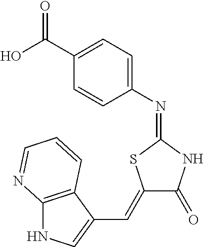 | (2Z,5Z)-2-(4- carboxyphenyl)azamethylene- 5-(1H-pyrrolo[2,3-b]pyridin-3- yl)methylene-1,3-thiazolidin- 4-one | 366.4 | ¹H NMR (300 MHz, DMSO-d6) δ = 8.38-8.33 (m, 3H), 7.95 (d, J = 8.7 Hz, 2H), 7.91 (s, 1H), 7.71 (m, 2H), 7.21 (dd, J = 4.7, 2.9 Hz, 2H) | A Method 6.019 |
| 5 | 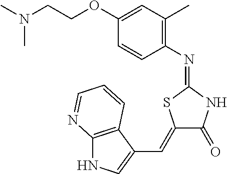 | (2Z,5Z)-2-{4-(2- dimethylamino)ethoxy-2- methylphenyl}azamethylene- 5-(1H-pyrrolo[2,3-b]pyridin-3- yl)methylene-1,3-thiazolidin- 4-one | 422.4 | ¹H NMR (300 MHz, DMSO-d6) δ = 8.30 (d, J = 6.5 Hz, 2H), 7.84 (s, 1H), 7.56 (s, 1H), 7.28 (dd, J = 6.2, 6.5 Hz, 1H), 6.92 (d, J = 8.4 Hz, 1H), 6.86 (s, 1H), 6.78 (dd, J = 2.5, 8.4 Hz, 1H), 4.03 (t, J = 5.4 Hz, 2H), 2.63 (t, J = 5.4 Hz, 2H), 2.22 (s, 6H), 2.12 (s, 3H) | A Method 5.78 |
| 6 | 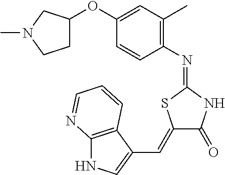 | (2Z,5Z)-2-{4-(1- methylpyrrolidin-3-yl)oxy-2- methylphenyl}azamethylene- 5-(1H-pyrrolo[2,3-b]pyridin-3- yl)methylene-1,3-thiazolidin- 4-one | 435.1 | ¹H NMR (300 MHz, DMSO-d6) δ = 8.29 (d, J = 6.9 Hz, 2H), 7.82 (s, 1H), 7.56 (s, 1H), 7.16 (dd, J = 5.1, 7.3 Hz, 1H), 6.90 (d, J = 8.4 Hz, 1H), 6.78 (s, 1H), 6.70 (dd, J = 2.5, 8.0 Hz, 1H), 4.82 (s, 1H), 2.83-2.51 (m, 3H), 2.41-2.27 (m, 2H), 2.25 (s, 3H), 2.11 (s, 3H), 2.77 (m, 1H) | A Method 5.788 |
| 7 | 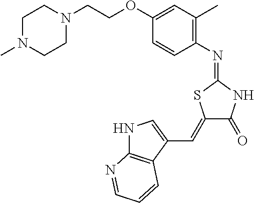 | (2Z,5Z)-2-[4-{2-(4- methylpiperazin-1-yl)}ethoxy- 2-methylphenyl]azamethylene- 5-(1H-pyrrolo[2,3-b]pyridin-3- yl)methylene-1,3-thiazolidin- 4-one | 477.3 | ¹H NMR (300 MHz, DMSO-d6) δ = 8.30 (d, J = 6.5 Hz, 2H), 7.84 (s, 1H), 7.56 (s, 1H), 7.17 (dd, J = 6.2, 6.5 Hz, 1H), 6.92 (d, J = 8.4 Hz, 1H), 6.87 (s, 1H), 6.77 (dd, J = 3.4, 8.4 Hz, 1H), 4.04 (t, J = 5.8 Hz, 2H), 2.66 (t, J = 5.4 Hz, 2H), 2.48 (m, 4H), 2.31 (m, 4H), 2.13 (s, 3H), 2.11 (s, 3H) | A Method 4.961 |
| 8 | 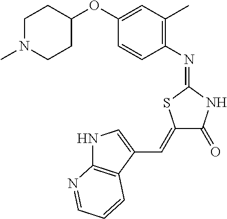 | (2Z,5Z)-2-{4-(1-methyl- piperidin-4-yl)oxy-2- methylphenyl}azamethylene- 5-(1H-pyrrolo[2,3-b]pyridin-3- yl)methylene-1,3-thiazolidin- 4-one | 448.4 | ¹H NMR (300 MHz, CD3OD) δ = 8.46 (s, 1H), 8.29 (m, 2H), 7.97 (s, 1H), 7.55 (s, 1H), 7.25 (dd, J = 4.7, 8.0 Hz, 1H), 7.03 (d, J = 8.0 Hz, 1H), 6.97 (d, J = 2.1 Hz, 1H), 6.90 (dd, J = 2.5, 8.4 Hz, 1H), 4.66 (s, 1H), 3.33 (m, 2H), 3.29 (m, 2H), 2.83 (s, 3H), 2.29 (s, 1H), 2.23 (s, 3H), 2.15 (m, 3H) | A Method 5.844 |
| 9 | 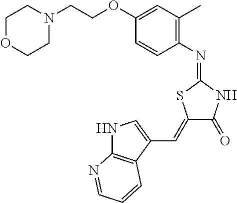 | (2Z,5Z)-2-{4-(2- morpholino)ethoxy-2- metlhylphenyl}azamethylene- 5-(1H-pyrrolo[2,3-b]pyridin-3- yl)methylene-1,3-thiazolidin- 4-one | 464.4 | ¹H NMR (300 MHz, DMSO-d6) δ = 8.30 (d, J = 6.5 Hz, 2H), 7.85 (s, 1H), 7.56 (s, 1H), 7.18 (dd, J = 6.2, 6.5 Hz, 1H), 6.92 (d, J = 8.7 Hz, 1H), 6.87 (d, J = 2.1 Hz, 1H), 6.78 (dd, J = 2.5, 8.4 Hz, 1H), 4.06 (t, J = 5.5 Hz, 2H), 3.57 (t, J = 4.3 Hz, 4H), 2.69 (t, J = 1.9 Hz, 2H), 2.47 (m, 4H), 2.12 (s, 3H) | A Method 6.760 |
| 10 | 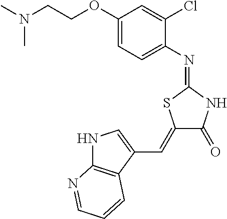 | (2Z,5Z)-2-{2-chloro-4-(2- dimethylamino)ethoxyphenyl} azamethylene-5-(1H- pyrrolo[2,3-b]pyridin-3- yl)methylene-1,3-thiazolidin- 4-one | 442.2 | ¹H NMR (400 MHz, DMSO-d6) δ = 8.34 (d, J = 6.4 Hz, 2H), 7.91 (s, 1H), 7.64 (s, 1H), 7.22 (dd, J = 4.4, 7.6 Hz, 1H), 7.16 (d, J = 2.0 Hz, 1H), 7.10 (d, J = 9.2 Hz, 1H), 6.97 (dd, J = 2.0, 8.8 Hz, 1H), 4.09 (t, J = 6.0 Hz, 2H), 2.67 (t, J = 5.6 Hz, 2H), 2.26 (s, 6H) | C Method 4.866 |
| 11 | 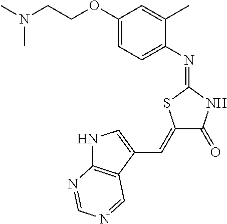 | (2Z,5Z)-2-{4-(2- dimethylamino)ethoxy-2- methylphenyl}azamethylene- 5-(7H-pyrrolo[2,3- d]pyrimidin-5-yl)methylene- 1,3-thiazolidin-4-one | 423.2 | ¹H NMR (400 MHz, DMSO-d6) δ = 9.38 (s, 1H), 8.85 (s, 1H), 7.92 (s, 1H), 7.69 (s, 1H), 6.95 (d, J = 8.0 Hz, 1H), 6.89 (d, J = 2.0 Hz, 1H), 6.80 (dd, J = 2.8, 8.8 Hz, 1H), 4.05 (t, J = 5.6 Hz, 2H), 2.65 (t, J = 4.8 Hz, 2H), 2.24 (s, 6H), 2.14 (s, 3H) | C Method 3.626 |
| 12 | 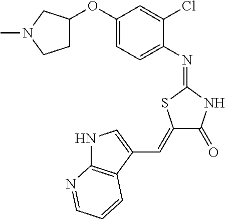 | (2Z,5Z)-2-{2-chloro-4-(1- methylpyrrolidin-3- yl)oxyphenyl}azamethylene- 5-(1H-pyrrolo[2,3-b]pyridin-3- yl)methylene-1,3-thiazolidin- 4-one | 454.1 | ¹H NMR (400 MHz, DMSO-d6) δ = 8.38 (s, 2H), 7.96 (s, 1H), 7.69 (s, 1H), 7.26 (s, 1H), 7.13 (m, 2H), 6.95 (d, J = 5.7 Hz, 1H), 4.96 (s, 1H), 2.90-2.75 (m, 3H), 2.57-2.40 (m, 2H), 2.38 (s, 3H), 1.89 (s, 1H) | C Method 4.91 |
| 13 | 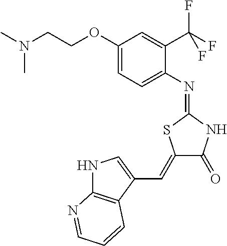 | (2Z,5Z)-2-{4-(2- dimethylamino)ethoxy-2- trifluoromethylphenyl} azamethylene-5-(1H-pyrrolo[2,3- b]pyridin-3-yl)methylene-1,3- thiazolidin-4-one | 476.2 | ¹H NMR (400 MHz, DMSO-d6) δ = 8.34 (t, J = 7.6 Hz, 2H), 7.91 (s, 1H), 7.56 (s, 1H), 7.29-7.17 (m, 4H), 4.15 (t, J = 5.6 Hz, 2H), 2.69 (t, J = 5.6 Hz, 2H), 2.27 (s, 6H) | C Method 3.179 |
| 14 | 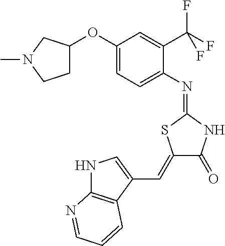 | (2Z,5Z)-2-{4-(1- methylpyrrolidin-3-yl)oxy-2- trifluoromethylphenyl} azamethylene-5-(1H-pyrrolo[2,3- b]pyridin-3-yl)methylene-1,3- thiazolidin-4-one | 488.5 | ¹H NMR (400 MHz, DMSO-d6) δ = 8.34 (t, J = 7.6 Hz, 2H), 7.91 (s, 1H), 7.65 (s, 1H), 7.24-7.16 (m, 4H), 4.92 (s, 1H), 2.82 (dd, J = 6.0, 10.0 Hz, 1H), 2.73 (m, 2H), 2.48- 2.38 (m, 2H), 2.35 (s, 3H), 1.85 (m, 1H) | C Method 4.553 |
| 15 | 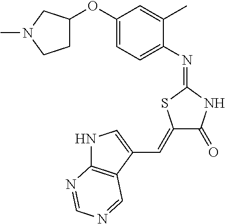 | (2Z,5Z)-2-{4-(1- methylpyrrolidin-3-yl)oxy-2- methylphenyl}azamethylene- 5-(7H-pyrrolo[2,3- d]pyrimidin-5-yl)methylene- 1,3-thiazolidin-4-one | 435.1 | ¹H NMR (300 MHz, DMSO-d6) δ = 9.61 (s, 1H), 9.03 (s, 1H), 7.97 (s, 1H), 7.82 (s, 1H), 6.97 (d, J = 8.4 Hz, 1H), 6.90 (s, 1H), 6.82 (d, J = 8.0 Hz, 1H), 5.14 (bs, 1H), 2.80- 2.82 (m, 4H), 2.61 (m, 1H), 2.14 (s, 3H), 1.63 (m, 1H) | A Method 5.232 |
| 16 | 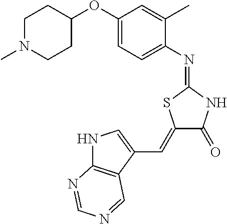 | (2Z,5Z)-2-{4-(1-methyl- piperidin-4-yl)oxy-2- methylphenyl}azamethylene- 5-(7H-pyrrolo[2,3- d]pyrimidin-5-yl)methylene- 1,3-thiazolidin-4-one | 449.2 | ¹H NMR (300 MHz, DMSO-d6) δ = 9.55 (s, 1H), 8.99 (s, 1H), 7.95 (s, 1H), 7.80 (s, 1H), 6.96-6.82 (m, 3H), 4.70 (s, 1H), 2.77 (m, 4H), 2.13 (s, 3H), 2.05 (m, 2H) | A Method 5.255 |
| 17 | 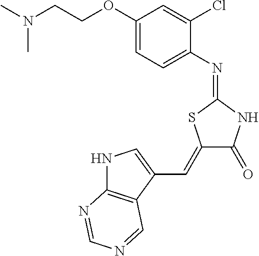 | (2Z,5Z)-2-{2-chloro-4-(2- dimethylamino)ethoxyphenyl} azamethylene-5-(7H- pyrrolo[2,3-d]pyrimidin-5- yl)methylene-1,3-thiazolidin- 4-one | 443.4 | ¹H NMR (400 MHz, DMSO-d6) δ = 9.39 (s, 1H), 8.86 (s, 1H), 7.95 (s, 1H), 7.73 (s, 1H), 7.15 (d, J = 2.0 Hz, 1H), 7.08 (d, J = 9.2 Hz, 1H), 6.92 (dd, J = 2.0, 8.8 Hz, 1H), 4.09 (t, J = 5.6 Hz, 2H), 2.67 (t, J = 5.6 Hz, 2H), 2.26 (s, 6H) | C Method 5.233 |
| 18 | 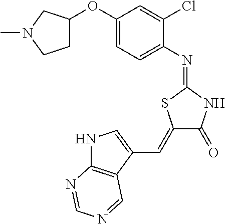 | (2Z,5Z)2-{2-chloro-4-(1- methylpyrrolidin-3- yl)oxyphenyl}azamethylene- 5-(7H-pyrrolo[2,3- d]pyrimidin-5-yl)methylene- 1,3-thiazolidin-4-one | 455.4 | ¹H NMR (400 MHz, DMSO-d6) δ = 9.39 (s, 1H), 8.86 (s, 1H), 7.96 (s, 1H), 7.74 (s, 1H), 7.08 (m, 2H), 6.90 (dd, J = 2.0, 8.8 Hz, 1H), 4.90 (bs, 1H), 2.80-2.58 (m, 3H), 2.40 (m, 1H), 2.35 (s, 3H), 1.78 (m, 1H) | C Method 3.980 |
| 19 | 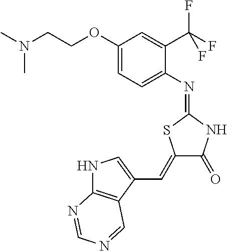 | (2Z,5Z)-2-{4-(2- dimethylamino)ethoxy-2- trifluoromethylphenyl} azamethylene-5-(7H-pyrrolo[2,3- d]pyrimidin-5-yl)methylene- 1,3-thiazolidin-4-one | 477.3 | ¹H NMR (400 MHz, DMSO-d6) δ = 9.39 (s, 1H), 8.85 (s, 1H), 7.95 (s, 1H), 7.73 (s, 1H), 7.26 (d, J = 9.6 Hz, 1H), 7.25 (s, 1H), 7.17 (d, J = 8.8, 1H), 4.15 (t, J = 4.8 Hz, 2H), 2.72 (t, J = 5.2 Hz, 2H), 2.29 (s, 6H) | C Method 3.613 |
| 20 | 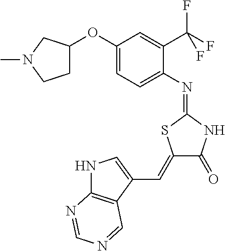 | (2Z,5Z)-2-{4-(1- methylpyrrolidin-3-yl)oxy-2- trifluoromethylphenyl} azamethylene-5-(7H-pyrrolo[2,3- d]pyrimidin-5-yl)methylene- 1,3-thiazolidin-4-one | 489.3 | ¹H NMR (400 MHz, DMSO-d6) δ = 9.40 (s, 1H), 8.86 (s, 1H), 7.97 (s, 1H), 7.75 (s, 1H), 7.23-7.17 (m, 3H), 4.99 (t, J = 7.6 Hz, 1H), 2.88- 2.73 (m, 3H), 2.46 (m, 1H), 2.35 (s, 3H), 1.76 (m, 1H) | C Method 4.367 |
| 21 | 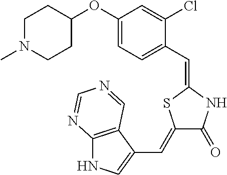 | (2Z,5Z)-2-{2-chloro-4-(1- methyl-piperidin-4- yl)oxyphenyl}azamethylene- 5-(7H-pyrrolo[2,3- d]pyrimidin-5-yl)methylene- 1,3-thiazolidin-4-one | 469.3 | ¹H NMR (400 MHz, DMSO-d6) δ = 9.39 (s, 1H), 8.86 (s, 1H), 7.95 (s, 1H), 7.73 (s, 1H), 7.15 (d, J = 2.8 Hz, 1H), 7.08 (d, J = 8.8 Hz, 1H), 6.98 (dd, J = 2.8, 8.4 Hz, 1H), 4.42 (bs, 1H), 2.71 (m, 2H), 2.31 (m, 2H), 2.27 (s, 3H), 1.98 (m, 2H), 1.68 (m, 2H) | C Method 4.356 |
| 22 | 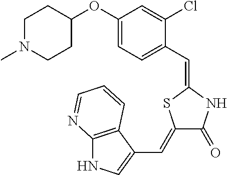 | (2Z,5Z)-2-{2-chloro-4-(1- methyl-piperidin-4- yl)oxyphenyl}azamethylene- 5-(1H-pyrrolo[2,3-b]pyridin-3- yl)methylene-1,3-thiazolidin- 4-one | 468.1 | ¹H NMR (300 MHz, DMSO-d6) δ = 8.31 (s, 1H), 8.29 (s, 1H), 7.86 (s, 1H), 7.61 (s, 1H), 7.18 (m, 1H), 7.12 (d, J = 2.5 Hz, 1H), 7.06 (d, J = 8.7 Hz, 1H), 6.94 (dd, J = 2.5, 8.4 Hz, 1H), 4.37 (bs, 1H), 2.63 (m, 2H), 2.22 (m, 2H), 2.18 (s, 3H), 1.95 (m, 2H), 1.64 (m, 2H) | A Method 5.725 |
| 23 | 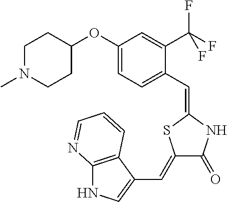 | (2Z,5Z)-2-{4-(1-methyl- piperidin-4-yl)oxy-2- trifluoromethylphenyl} azamethylene-5-(1H-pyrrolo[2,3- b]pyridm-3-yl)methylene-1,3- thiazolidin-4-one | 502.2 | ¹H NMR (300 MHz, DMSO-d6) δ = 8.32 (m, 2H), 7.87 (s, 1H), 7.62 (s, 1H), 7.27 (d, J = 8.0 Hz, 1H), 7.20-7.12 (m, 3H), 4.45 (bs, 1H), 2.64 (m, 2H), 2.24 (m, 2H), 2.20 (s, 3H), 1.92 (m, 2H), 1.66 (m, 2H) | A Method 5.846 |

### [Cdc7 Protein Kinase Inhibitory Action of (Di)Azaindole Derivatives]

Cdc7 protein kinase inhibitory action of the (di)azaindole derivatives shown in Table 4 was evaluated according to the following operations.

As a substrate for evaluating the enzymatic activity, mouse MCM2-4(his)-6-7 (his) conjugated protein was synthesized. First, the following genes were cloned into a pAcUW31 vector (Pharmingen).

Mouse MCM2 (Genbank/EMBL No. D86725);

N-His-mouse MCM4 (Genbank/EMBL No. D26089, a His6 tag was added to an N-terminal);

Mouse MCM6 (Genbank/EMBL No. D86726), and

N-His-mouse MCM7 (Genbank/EMBL No. D26091, a His6 tag was added to an N-terminal).

The Mouse MCM2 gene and the N-His-mouse MCM7 gene were inserted into the BamHI site and the EcoRI site of the pAcUW31 vector (Mom2-7 Vector), respectively. The mouse MCM6 gene and the N-His-mouse MCM4 gene were inserted into the BamHI site and the EcoRI site of the pAcUW31 vector (MGm-4-6 Vector), respectively. Then, in order to produce a recombinant mouse MCM2-7 (his) and mouse MCM4 (his)-6 Baculovirus, BaculoGold AoNPV Baculovirus DNA (BD/Pharmingen) and an Mcm2-7 Vector or an Mcm-4-6 Vector were cotransfected to SD insect cells. The obtained Mcm2-7 and Mom-4-6 virus were coinfected to Hi5 insect cells. Thereby, a mouse MCM2-4 (his)-6-7 (his) conjugated protein was obtained. Purified mouse MCM2-4 (his)-6-7 (his) conjugated protein (0.5 μg) was used as a substrate.

The substrate (mouse MCM2-4(his)-6-7(his) conjugated protein) (0.5 μg) and kinase enzyme (human Cdc7/human ASK conjugated protein, CARNA BIOSCIENCES) (0.1 μg) were mixed in a microtube. A kinase reaction buffer containing 0.1 μL of γ-³²P ATP (Perkin Elmer) was added as a tracer into the same microtube. The composition of the kinase buffer includes the followings.

40 mM HEPES-KOH buffer pH 7.6,

0.5 mM EDTA,

0.5 mM EGTA,

1 mM β-glycerophosphate,

1 mM NaF,

2 mM Dithiothreitol

0.1 mM ATP

Furthermore, 0.5 μL of each of the test compounds ((di)azaindole derivatives 10 μM, 100 μM, and 1000 μM, respectively) was added and reacted at 30° C. for 45 minutes. After the reaction, the substrate was separated by SDS-PAGE method, and protein was stained by the silver staining method (2D-silver staining reagent H “Daiichi,” Daiichi Pure Chemicals Co., Ltd.).

The radioactivity of ³²P-labeled MCM2 protein was detected by autoradiography, and the radioactivity of the band of MCM2 protein was measured by a liquid scintillation counter (LSC-6100, ALOKA). As to the Cdc7-ASK kinase activity, the radioactivity at the time when the each of the test compounds was added was calculated in percent when the radioactivity at the time when DMSO (0.5 μL) was added instead of the test compound (0.5 μL) that had been added immediately before the reaction was made to be 100%. Based on the obtained results, IC 50 value of each test compound was calculated. IC50 with respect to Cdc7-ASK kinase of the main (di)azaindole derivatives shown in Table 5 was about 0.001 μM to 0.008 μM.

TABLE 5

| | | Activity (uM) |
|---|---|---|
| Compound | | ELISA |
| No. | Structure | (IC50) |
| 1 | 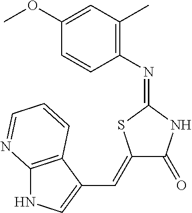 | 0.0012 |
| 2 | 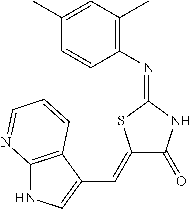 | 0.001 |
| 3 | 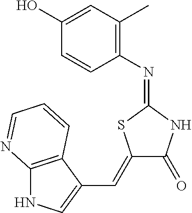 | 0.0014 |
| 4 | 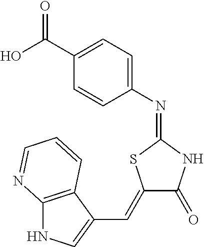 | 0.018 |
| 5 | 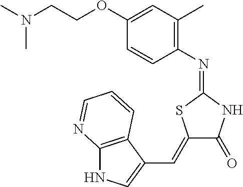 | 0.0019 |
| 6 | 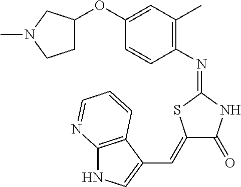 | 0.003 |
| 7 | 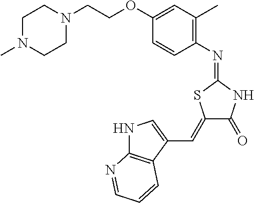 | 0.0021 |
| 8 | 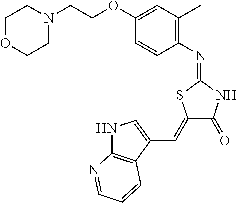 | 0.0011 |
| 9 | 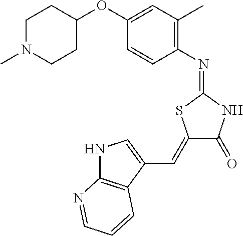 | 0.0017 |
| 10 | 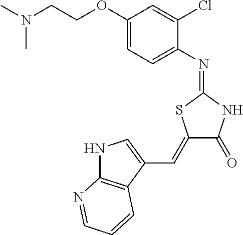 | 0.002 |
| 11 | 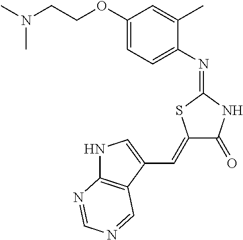 | 0.005 |
| 12 | 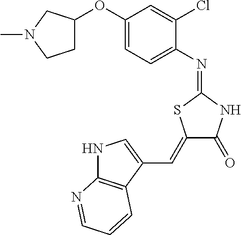 | 0.001 |
| 13 | 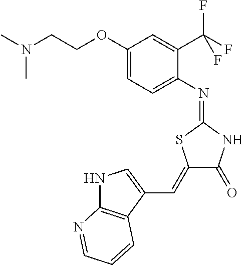 | 0.003 |
| 14 | 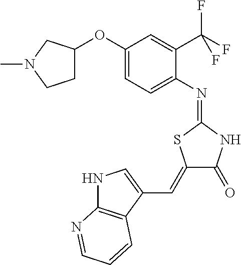 | 0.002 |
| 15 | 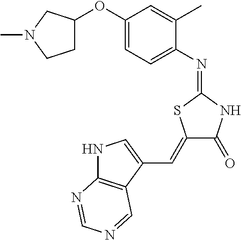 | 0.008 |
| 16 | 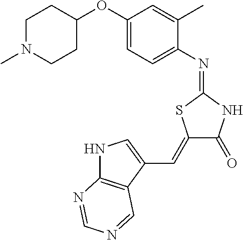 | 0.006 |
| 17 | 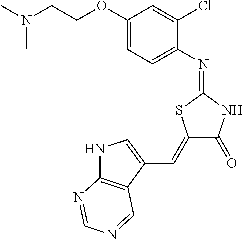 | 0.002 |
| 18 | 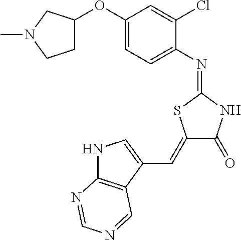 | 0.002 |
| 19 | 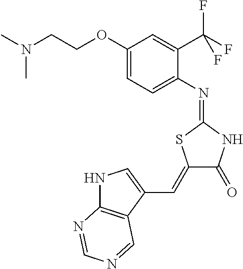 | 0.006 |
| 20 | 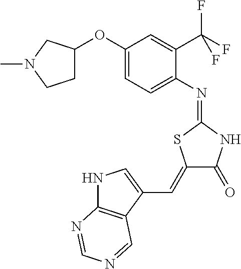 | 0.007 |
| 21 | 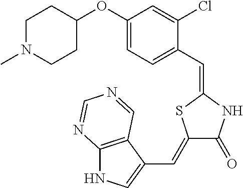 | 0.003 |
| 22 | 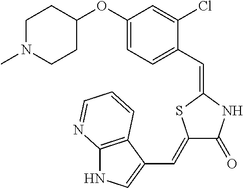 | 0.001 |
| 23 | 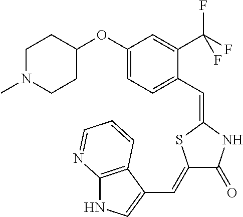 | 0.003 |

### INDUSTRIAL APPLICABILITY

A novel (di)azaindole derivative provided by the present invention is useful as a Cdc7 protein kinase inhibitor. The Cdc7 protein kinase is a molecule that plays an important role in DNA replication. Therefore, compounds that inhibit the action of Cdc7 protein kinase can be used as a suppressing agent of cell proliferation.

1. A compound represented by the following formula (I), a geometric isomer or a tautomer thereof, or a salt, a hydrate, or a solvate thereof: 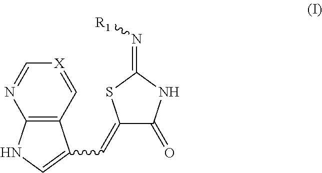
wherein
X is CH or N;
R₁ is selected from the group consisting of a straight or branched chain lower alkyl group, a cycloalkyl group that may have a substituent, an aryl group that may have a substituent, an arylalkyl group that may have a substituent, a non-aromatic heterocyclic group that may have a substituent, and a heteroaryl group that may have a substituent, or is a condensed ring group that may have a substituent; and
a wavy line, independently for each occurrence, denotes trans (E-form), cis (Z-form) or a mixture (mixed product) thereof.

2. The compound, a geometric isomer and a tautomer thereof, as well as a salt, a hydrate, or a solvate thereof according to claim 1,
wherein R₁ is an aryl group or a heteroaryl group which may be substituted with one to three groups independently selected from the following Group B;
Group B:
a straight or branched chain lower alkyl group which may be substituted with a group selected from the group consisting of one to three halogen atoms, a hydroxyl group, an amino group substituted with one or two lower alkyl groups and a non-aromatic heterocyclic group;
a lower alkoxy group;
a hydroxyl group;
a halogen group;
a nitro group;
an amino group that may be substituted with one or two lower alkyl groups;
a lower alkylcarbonylamino group;
a group represented by a formula: —(CH₂)ₖCOOH, wherein k is 0 to 2;
a group represented by a formula: —O—R₂—R₃, wherein R₂ is a single bond, a lower alkylene group or a cycloalkylene group, or a non-aromatic heterocyclic group that may be substituted with a lower alkyl group; and R₃ is a group selected from a hydroxyl group; a carboxyl group; a lower alkoxy group; a lower alkoxycarbonyl group; an amino group substituted with two lower alkyl groups or with one lower alkyl group and one lower alkoxy carbonyl group; and a non-aromatic heterocyclic group that may be substituted with a lower alkyl group; and
a group represented by a formula: —CON(R₄)[(CH₂)ₘ—R₅], wherein m is 0 to 2, R₄ is a hydrogen atom or a lower alkyl group, and R₅ is an amino group that is substituted with one or two lower alkyl groups.

3. The compound, a geometric isomer and a tautomer thereof, as well as a salt, a hydrate, or a solvate according to claim 2, wherein the aryl group or the heteroaryl group is a phenyl group, a naphthyl group, an indolyl group, an indazolyl group, a quinolyl group, a benzimidazolyl group or a benzotriazolyl group.

4. The compound, a geometric isomer and a tautomer thereof as well as a salt, a hydrate, or a solvate thereof according to claim 1, wherein the R₁ is a benzyl group having a substituent, and a benzene ring of the benzyl group is substituted with halogen, a lower alkyl group that may be substituted with one to three halogen atoms or a lower alkoxy group, or methylene of the benzyl group is substituted with one or two lower alkyl.

5. The compound, a geometric isomer and a tautomer thereof, as well as a salt, a hydrate, or a solvate thereof according to claim 1, wherein the R₁ is an indanyl group or a 1,3-benzodioxolyl group.

6. The compound, a geometric isomer and a tautomer thereof as well as a salt, a hydrate, or a solvate thereof according to claim 1, wherein the R₁ is a phenyl group having a substituent.

7. The compound, a geometric isomer and a tautomer thereof, as well as a salt, a hydrate, or a solvate thereof according to claim 1, wherein the R₁ is a phenyl group having a substituent, and the substituent is a group represented by a formula: —O—R₂—R₃, wherein R₂ is a single bond, a lower alkylene group or a cycloalkylene group, or a non-aromatic heterocyclic group that may be substituted with a lower alkyl group; and R₃ is a group selected from a hydroxyl group; a carboxyl group; a lower alkoxy group; a lower alkoxycarbonyl group; an amino group substituted with two lower alkyl groups, or with one lower alkyl group and one lower alkoxy carbonyl group; and a non-aromatic heterocyclic group that may be substituted with a lower alkyl group.

8. A pharmaceutical composition comprising a compound, a geometric isomer and a tautomer thereof, or a salt, a hydrate, or a solvate thereof according to any one of claims 1 to 7, and a pharmaceutically acceptable carrier.

9. A process for producing a compound represented by the following formula (I), which comprises reacting a compound represented by the following formula (IIA) or (IIB) with a compound represented by the following formula (III): 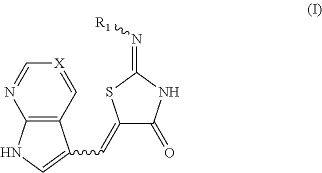
wherein R₁ is selected from the group consisting of a hydrogen atom, a straight or branched chain lower alkyl group, halogen, a hydroxyl group, an amino group that may have a substituent and a non-aromatic heterocyclic group that may have a substituent;
R₂ is a hydrogen atom or a straight or branched chain lower alkyl group;
R₃ is selected from the group consisting of a straight or branched chain lower alkyl group; a cycloalkyl group that may have a substituent, an aryl group that may have a substituent, an arylalkyl group that may have a substituent, a non-aromatic heterocyclic group that may have a substituent, and a heteroaryl group that may have a substituent, or is a condensed ring group that may have a substituent; and
a wavy line, independently for each occurrence, denotes trans (E-form), cis (Z-form) or a mixture (mixed product) thereof: 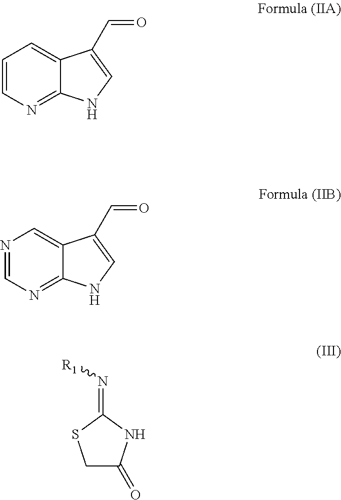
wherein R₁ is the same as the R₁ in the above formula (I).

10. The compound, a geometric isomer and a tautomer thereof, or a salt, a hydrate, or a solvate thereof according to claim 1, selected from the group consisting of (2Z,5Z)-2-(4-methoxy-2-methylphenyl)azamethylene-5-(1H-pyrrolo[2,3-b]pyridin-3-yl)methylene-1,3-thiazolidin-4-one, (2Z,5Z)-2-(2,4-dimethylphenyl)azamethylene-5-(1H-pyrrolo[2,3-b]pyridin-3-yl)methylene-1,3-thiazolidin-4-one, (2Z,5Z)-2-(4-hydroxy-2-methylphenyl)azamethylene-5-(1H-pyrrolo[2,3-b]pyridin-3-yl)methylene-1,3-thiazolidin-4-one, (2Z,5Z)-2-(4-carboxyphenyl)azamethylene-5-(1H-pyrrolo[2,3-b]pyridin-3-yl)methylene-1,3-thiazolidin-4-one, (2Z,5Z)-2-{4-(2-dimethylamino)ethoxy-2-methylphenyl}azamethylene-5-(1H-pyrrolo[2,3-b]pyridin-3-yl)methylene-1,3-thiazolidin-4-one, (2Z,5Z)-2-{4-(1-methylpyrrolidin-3-yl)oxy-2-methylphenyl}azamethylene-5-(1H-pyrrolo[2,3-b]pyridin-3-yl)methylene-1,3-thiazolidin-4-one, (2Z,5Z)-2-[4-{2-(4-methylpiperazin-1-yl)}ethoxy-2-methylphenyl]azamethylene-5-(1H-pyrrolo[2,3-b]pyridin-3-yl)methylene-1,3-thiazolidin-4-one, (2Z,5Z)-2-{4-(1-methyl-piperidin-4-yl)oxy-2-methylphenyl}azamethylene-5-(1H-pyrrolo[2,3-b]pyridin-3-yl)methylene-1,3-thiazolidin-4-one, (2Z,5Z)-2-{4-(2-morpholino)ethoxy-2-methylphenyl}azamethylene-5-(1H-pyrrolo[2,3-b]pyridin-3-yl)methylene-1,3-thiazolidin-4-one, (2Z,5Z)-2-{2-chloro-4-(2-dimethylamino)ethoxyphenyl}azamethylene-5-(1H-pyrrolo[2,3-b]pyridin-3-yl)methylene-1,3-thiazolidin-4-one, (2Z,5Z)-2-{4-(2-dimethylamino)ethoxy-2-methylphenyl}azamethylene-5-(7H-pyrrolo[2,3-d]pyrimidin-5-yl)methylene-1,3-thiazolidin-4-one, (2Z,5Z)-2-{2-chloro-4-(1-methylpyrrolidin-3-yl)oxyphenyl}azamethylene-5-(1H-pyrrolo[2,3-b]pyridin-3-yl)methylene-1,3-thiazolidin-4-one, (2Z,5Z)-2-{4-(2-dimethylamino)ethoxy-2-trifluoromethylphenyl}azamethylene-5-(1H-pyrrolo[2,3-b]pyridin-3-yl)methylene-1,3-thiazolidin-4-one, (2Z,5Z)-2-{4-(1-methylpyrrolidin-3-yl)oxy-2-trifluoromethylphenyl}azamethylene-5-(1H-pyrrolo[2,3-b]pyridin-3-yl)methylene-1,3-thiazolidin-4-one, (2Z,5Z)-2-{4-(1-methylpyrrolidin-3-yl)oxy-2-methylphenyl}azamethylene-5-(7H-pyrrolo[2,3-d]pyrimidin-5-yl)methylene-1,3-thiazolidin-4-one, (2Z,5Z)-2-{4-(1-methyl-piperidin-4-yl)oxy-2-methylphenyl}azamethylene-5-(7H-pyrrolo[2,3-d]pyrimidin-5-yl)methylene-1,3-thiazolidin-4-one, (2Z,5Z)-2-{2-chloro-4-(2-dimethylamino)ethoxyphenyl}azamethylene-5-(7H-pyrrolo[2,3-d]pyrimidin-5-yl)methylene-1,3-thiazolidin-4-one, (2Z,5Z)-2-{2-chloro-4-(1-methylpyrrolidin-3-yl)oxyphenyl}azamethylene-5-(7H-pyrrolo[2,3-d]pyrimidin-5-yl)methylene-1,3-thiazolidin-4-one, (2Z,5Z)-2-{4-(2-dimethylamino)ethoxy-2-trifluoromethylphenyl}azamethylene-5-(7H-pyrrolo[2,3-d]pyrimidin-5-yl)methylene-1,3-thiazolidin-4-one, (2Z,5Z)-2-{4-(1-methylpyrrolidin-3-yl)oxy-2-trifluoromethylphenyl}azamethylene-5-(7H-pyrrolo[2,3-d]pyrimidin-5-yl)methylene-1,3-thiazolidin-4-one, (2Z,5Z)-2-{2-chloro-4-(1-methyl-piperidin-4-yl)oxyphenyl}azamethylene-5-(7H-pyrrolo[2,3-d]pyrimidin-5-yl)methylene-1,3-thiazolidin-4-one, (2Z,5Z)-2-{2-chloro-4-(1-methyl-piperidin-4-yl)oxyphenyl}azamethylene-5-(1H-pyrrolo[2,3-b]pyridin-3-yl)methylene-1,3-thiazolidin-4-one, and (2Z,5Z)-2-{4-(1-methyl-piperidin-4-yl)oxy-2-trifluoromethylphenyl}azamethylene-5-(1H-pyrrolo[2,3-b]pyridin-3-yl)methylene-1,3-thiazolidin-4-one.
